(19) United States
(12) Patent Application Publication
(10) Pub. No.: US 2002/0004490 A1
(43) Pub. Date: Jan. 10, 2002

(54) Antisense modulation of Fas mediated signaling

(76) Inventors: Nicholas M. Dean, Olivenhain, CA (US); Eric G. Marcusson, San Diego, CA (US); Jacqueline Wyatt, Encinitas, CA (US); Hong Zhang, Carlsbad, CA (US)

Correspondence Address: Jane Massey Licata, Licata & Tyrrell P.C., 66 E. Main Street, Marlton, NJ, 08053, US

(21) Appl. No.: 09/802,669

(22) Filed: Mar. 9, 2001

Related U.S. Application Data
(63) Continuation-in-part of application No. 09/665,615, filed on Sep. 18, 2000.
Application No. 09/665,615 is a continuation-in-part of application No. 09/290,640, filed on Apr. 12, 1999, now Pat. No. 6,204,055.

(51) Int. Cl. A61K 48/00; C07H 21/04

(52) U.S. Cl. 514/44; 536/23.1

(57) ABSTRACT

Compounds, compositions and methods are provided for inhibiting Fas mediated signaling. The compositions comprise antisense compounds targeted to nucleic acids encoding Fas, FasL and Fap-1. Methods of using these antisense compounds for inhibition of Fas, FasL and Fap-1 expression and for treatment of diseases, particularly autoimmune and inflammatory diseases and cancers, associated with overexpression or constitutive activation of Fas, FasL or Fap-1 are provided.

This application is a continuation-in-part of U.S. patent application Ser. No. 09/665,615 filed Sep. 18, 2000, which is a continuation-in-part of U.S. patent application Ser. No. 09/290,640 filed Apr. 12, 1999.

### FIELD OF THE INVENTION

This invention relates to compositions and methods for modulating expression of the human Fas, FasL and Fap-1 genes, which encode proteins involved in Fas mediated signal transduction and are implicated in disease. This invention is also directed to methods for inhibiting Fas, FasL or Fap-1-mediated signal transduction; these methods can be used diagnostically or therapeutically. Furthermore, this invention is directed to treatment of conditions associated with expression of the human Fas, FasL or Fap-1 genes.

### BACKGROUND OF THE INVENTION

The Fas ligand (FasL, also CD95L or Apo-1L) belongs to the tumor necrosis factor (TNF) family. It associates with the Fas receptor (Fas, also CD95 or Apo-1). Both function primarily as membrane-bound cell-surface proteins. The interaction between Fas and FasL is a key regulator of apoptosis within the immune system. Binding of FasL by Fas triggers apoptosis. Since both Fas and FasL are typically membrane-bound, cells expressing either Fas or FasL generally must come into contact with cells expressing the other in order to induce cell death (Rowe, P. M., Lancet, 1996, 347, 1398). Under normal conditions, expression of the FasL is generally limited to activated T cells and macrophages. Fas is expressed in a variety of lymphoid and non-lymphoid cells including thymus, liver, heart and kidney (Watanabe-Fukunaga, R., et al., J. Immunol., 1992, 148, 1274-1279).

Expression of FasL is involved in a number of cancers, including lymphomas, melanoma (Hahne, M., et al., Science, 1996, 274, 1363-1366), colon, hepatic and lung carcinomas and astrocytomas (Saas, P., et al., J. Clin. Invest., 1997, 99, 1173-1178). It is thought that FasL expression by tumor cells is a mechanism by which they escape killing by the immune system and instead enables them to kill immune cells possessing Fas receptor on their surfaces (Walker, P. R., et al., J. Immunol., 1997, 158, 4521-4524).

Fas and FasL are also involved in other diseases, including autoimmune and inflammatory diseases. These include Hashimoto's thyroiditis (Giordano, C., et al., Science, 1997, 275, 1189-1192), hepatitis (Kondo, T., et al., Nat. Med., 1997, 3, 409-413), diabetes (Chervonsky, A. V., et al., Cell, 1997, 89, 17-24), myasthenia gravis (Moulian, N., et al., Blood, 1997, 89, 3287-3295), ulcerative colitis (Strater, J., et al., Gastroenterology, 1997, 113, 160-167), autoimmune gastritis (Nishio, A., et al., Gastroenterology 1996, 111, 959-967), Sjogren's syndrome (Kong, L., et al., Arthritis Rheum., 1997, 40, 87-97) and HIV infection (Sieg, S., et al., Proc. Natl. Acad. Sci (USA), 1997, 94, 5860-5865).

Fap-1 (Fas associated protein 1 or protein tyrosine phosphatase (PTP-BAS, type 1)) is a tyrosine phosphatase that binds with a negative regulatory element of Fas (Sato, T., et al., Science, 1995, 268, 411-415). It also is an inhibitor of Fas-mediated apoptosis and an important component of Fas mediated signaling. The presence of Fap-1 in tumor cell lines also correlated with resistance to Fas antibody. Takahashi, M. et al. (Gan To Kagaku Ryoho, 1997, 24, 222-228) found that Fap-1 was expressed in many colon cancer cell lines, but not in normal colon cells.

Several approaches have been used to study the interaction between Fas and FasL and could potentially be used for therapeutic purposes. One way to disrupt the balance (altered or normal) between Fas and FasL is to provide additional amounts of one of them. This approach has been used with soluble Fas by Kondo, T., et al. (Nature Med., 1997, 3, 409-413) to prevent hepatitis in a transgenic mouse model and Cheng, J., et al. (Science, 1994, 263, 1759-1762) to inhibit Fas-mediated apoptosis in systemic lupus erythematosus. Arai, H., et al. (Proc. Natl. Acad. Sci. USA, 1997, 94, 13862-13867) used a somewhat different approach to increase FasL. An adenoviral expression vector containing FasL was used to infect tumor cells. The increased levels of FasL induced apoptosis and caused tumor regression.

Portions of these proteins could also be used. It was found that the three C-terminal amino acids of Fas were necessary and sufficient for binding to Fap-1 (Yanagisawa, J., et al., J. Biol. Chem., 1997, 272, 8539-8545). Introduction of this peptide into a colon cancer cell line induced Fas-mediated apoptosis.

Monoclonal antibodies to Fas have been used extensively to induce apoptosis. Anti-Fas antibodies resulted in tumor regression in B cell tumors (Trauth B. C., et al., Science, 1989, 245, 301-305), adult T-cell leukemia (Debatin, K. M., et al., Lancet, 1990, 335, 497-500), gliomas (Weller, M., et al., J. Clin. Invest., 1994, 94, 954-964), and colorectal cancer (Meterissian, S. H., Ann. Surg. Oncol., 1997, 4, 169-175). Antibodies to Fas also killed HIV infected cells (Kobayashi, N., et al., Proc. Natl. Acad. Sci USA, 1990, 87, 9620-9624). Monoclonal antibodies have been used in combination with chemotherapeutic drugs to overcome drug resistance (Morimoto, H., et al., Cancer Res., 1993, 53, 2591-2596), Nakamura, S., et al., Anticancer Res., 1997, 17, 173-179) and Wakahara, Y., et al., Oncology, 1997, 54, 48-54).

Chemical agents have been used to inhibit FasL expression (Yang, Y., et al., J. Exp. Med., 1995, 181, 1673-1682). Retinoic acid and corticosteroids inhibit the up-regulation of FasL.

An antisense RNA approach, involving the antisense expression of a significant portion of a gene, has been used to modulate expression of Fas and Fap-1. Herr, I. et al. (EMBO J., 1997, 16, 6200-6208) expressed a 360 bp fragment of Fas in the antisense orientation to inhibit apoptosis. Freiss, G. et al. (Mol. Endocrinol., 1998, 12, 568-579) expressed a greater than 600 bp fragment of Fap-1 to inhibit Fap-1 expression.

Oligonucleotides have also been used to modulate expression of FasL. A bifunctional ribozyme targeted to both perforin and FasL was designed to treat graft-versus-host disease (Du, Z., et al., Biochem. Biophys. Res. Commun., 1996 226, 595-600). Antisense oligonucleotides have been used against both Fas and FasL. Yu, W. et al. (Cancer Res., 1999, 59, 953-961) used an oligonucleotide targeted to the translation initiation site of human Fas to reduce Fas mediated signaling in breast cancer cells. Lee, J., et al. (Endocrinology, 1997, 138, 2081-2088) used an oligonucleotide targeted to the translation initiation region of rat FasL to show that Fas system regulates spermatogenesis. Turley, J. M., et al. (Cancer Res., 1997, 57, 881-890) used an oligonucleotide targeted to the translation initiation region of human FasL to show that the Fas system was involved in Vitamin E succinate mediated apoptosis of human breast cancer cells. O'Connell, J., et al. (J. Exp. Med., 1996, 184, 1075-1082) used a model involving Jurkat T cells and SW620, a colon cancer cell line. The presence of FasL on SW620 causes apoptosis of Jurkat cells which possess the Fas receptor. Antisense oligonucleotides to either the FasL on SW620 or Fas on Jurkat cells could prevent apoptosis of the Jurkat cells. Oligonucleotides were designed to target sequences toward the 3′ end of the coding region.

There remains a long-felt need for improved compositions and methods for inhibiting Fas, FasL and Fap-1 gene expression.

### SUMMARY OF THE INVENTION

The present invention provides antisense compounds, including antisense oligonucleotides, which are targeted to nucleic acids encoding Fas, FasL and Fap-1 and are capable of modulating Fas mediated signaling. The present invention also provides chimeric oligonucleotides targeted to nucleic acids encoding human Fas, FasL and Fap-1. The compounds and compositions of the invention are believed to be useful both diagnostically and therapeutically, and are believed to be particularly useful in the methods of the present invention. The present invention also comprises methods of modulating the Fas mediated signaling, in cells and tissues, using the antisense compounds of the invention. Methods of inhibiting Fas, FasL and Fap-1 expression are provided; these methods are believed to be useful both therapeutically and diagnostically. These methods are also useful as tools, for example, for detecting and determining the role of Fas, FasL and Fap-1 in various cell functions and physiological processes and conditions and for diagnosing conditions associated with expression of Fas, FasL or Fap-1.

The present invention also comprises methods for diagnosing and treating autoimmune and inflammatory diseases, particularly hepatitis, and cancers, including those of the colon, liver and lung, and lymphomas. These methods are believed to be useful, for example, in diagnosing Fas, FasL and Fap-1-associated disease progression. These methods employ the antisense compounds of the invention. These methods are believed to be useful both therapeutically, including prophylactically, and as clinical research and diagnostic tools.

In accordance with the present invention, compositions for inhibiting allograft rejection, ischemia reperfustion injury and apoptosis are provided. These compositions comprise an antisense oligonucleotide which is targeted to a nucleic acid sequence encoding Fas.

Also in accordance with the present invention, methods of inhibiting allograft rejection, ischemia reperfusion injury and apoptosis are provided which comprise treating an allograft recipient with an antisense oligonucleotide which is targeted to a nucleic acid sequence encoding Fas. In addition, the allograft may be treated with the antisense oligonucleotide, ex vivo.

### DETAILED DESCRIPTION OF THE INVENTION

Fas, FasL and Fap-1 play important roles in signal transduction. Overexpression and/or constitutive activation of Fas, FasL or Fap-1 is associated with a number of autoimmune and inflammatory diseases, and cancers. As such, these proteins involved in signal transduction represent attractive targets for treatment of such diseases. In particular, modulation of the expression of Fas, FasL or Fap-1 may be useful for the treatment of diseases such as hepatitis, colon cancer, liver cancer, lung cancer and lymphomas.

The present invention employs antisense compounds, particularly oligonucleotides, for use in modulating the function of nucleic acid molecules encoding Fas, FasL and Fap-1, ultimately modulating the amount of Fas, FasL or Fap-1 produced. This is accomplished by providing oligonucleotides which specifically hybridize with nucleic acids, preferably mRNA, encoding Fas, FasL or Fap-1.

This relationship between an antisense compound such as an oligonucleotide and its complementary nucleic acid target, to which it hybridizes, is commonly referred to as “antisense”. “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the targets are nucleic acids encoding Fas, FasL or Fap-1; in other words, a gene encoding Fas, FasL or Fap-1, or mRNA expressed from the Fas, FasL or Fap-1 gene. mRNA which encodes Fas, FasL or Fap-1 is presently the preferred target. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed MRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding Fas, FasL or Fap-1, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region,” “AUG region” and “translation initiation codon region” refer to a portion of such an mRNA or gene that encompasses from about 25 to about 50 contiguous nucleotides in either direction (i.e., 5′ or 3′) from a translation initiation codon. This region is a preferred target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a preferred target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an MRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns”, which are excised from a pre-mRNA transcript to yield one or more mature MRNA. The remaining (and therefore translated) regions are known as “exons” and are spliced together to form a continuous mRNA sequence. mRNA splice sites, i.e., exon-exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

Once the target site or sites have been identified, oligonucleotides are chosen which are sufficiently complementary to the target, i.e., hybridize sufficiently well and with sufficient specificity, to give the desired modulation.

“Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them.

“Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The overall effect of interference with mRNA function is modulation of expression of Fas, FasL or Fap-1. In the context of this invention “modulation” means either inhibition or stimulation; i.e., either a decrease or increase in expression. This modulation can be measured in ways which are routine in the art, for example by Northern blot assay of mRNA expression, or reverse transcriptase PCR, as taught in the examples of the instant application or by Western blot or ELISA assay of protein expression, or by an immunoprecipitation assay of protein expression. Effects on cell proliferation or tumor cell growth can also be measured, as taught in the examples of the instant application. Inhibition is presently preferred.

The oligonucleotides of this invention can be used in diagnostics, therapeutics, prophylaxis, and as research reagents and in kits. Since the oligonucleotides of this invention hybridize to nucleic acids encoding Fas, FasL or Fap-1, sandwich, calorimetric and other assays can easily be constructed to exploit this fact. Provision of means for detecting hybridization of oligonucleotide with the Fas, FasL or Fap-1 genes or mRNA can routinely be accomplished. Such provision may include enzyme conjugation, radiolabelling or any other suitable detection systems. Kits for detecting the presence or absence of Fas, FasL or Fap-1 may also be prepared.

The present invention is also suitable for diagnosing abnormal inflammatory states or certain cancers in tissue or other samples from patients suspected of having an autoimmune or inflammatory disease such as hepatitis or cancers such as those of the colon, liver or lung, and lymphomas. A number of assays may be formulated employing the present invention, which assays will commonly comprise contacting a tissue sample with an oligonucleotide of the invention under conditions selected to permit detection and, usually, quantitation of such inhibition. In the context of this invention, to “contact” tissues or cells with an oligonucleotide or oligonucleotides means to add the oligonucleotide(s), usually in a liquid carrier, to a cell suspension or tissue sample, either in vitro or ex vivo, or to administer the oligonucleotide(s) to cells or tissues within an animal.

The oligonucleotides of this invention may also be used for research purposes. Thus, the specific hybridization exhibited by the oligonucleotides may be used for assays, purifications, cellular product preparations and in other methodologies which may be appreciated by persons of ordinary skill in the art.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The antisense compounds in accordance with this invention preferably comprise from about 5 to about 50 nucleobases. Particularly preferred are antisense oligonucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2=, 3=or 5=hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500).

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃) —CH₂—,—CH₂—N(CH₃)—N(CH₃)—CH₂— and —O—N(CH₃)—CH₂CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl, O-alkyl-O-alkyl, O-, S-, or N-alkenyl, or O-, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]mCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2=position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 1995, 78, 486-504) i.e., an alkoxyalkoxy group. A further preferred modification includes 2′-dimethylaminooxyethoxy, i.e., a O(CH₂) ₂ON(CH₃)₂ group, also known as 2′-DMAOE, and 2′-dimethylamino-ethoxyethoxy (2′-DMAEOE), i.e., 2′-O—CH₂—O—CH₂—N(CH₂)₂.

Other preferred modifications include 2¹-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 31 position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Chapter 15, Antisense Research and Applications 1993, pages 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications 1993, CRC Press, Boca Raton, pages 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-S-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther. 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941.

The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids. Ribozymes are not comprehended by the present invention.

Examples of chimeric oligonucleotides include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 31 “wings”) are modified to have greater affinity for the target RNA molecule but are unable to support nuclease activity (e.g., fluoro- or 2′-O-methoxyethyl- substituted). Chimeric oligonucleotides are not limited to those with modifications on the sugar, but may also include oligonucleosides or oligonucleotides with modified backbones, e.g., with regions of phosphorothioate (P═S) and phosphodiester (P═O) backbone linkages or with regions of MMI and P═S backbone linkages. Other chimeras include “wingmers,” also known in the art as “hemimers,” that is, oligonucleotides with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl- substituted), or vice-versa. In one embodiment, the oligonucleotides of the present invention contain a 2′-O-methoxyethyl (2′-O—CH₂CH₂OCH₃) modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (—O—CH₂CH₂OCH₃) modification. oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also preferred. Chimeric oligonucleotides comprising one or more such modifications are presently preferred.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is well known to use similar techniques to prepare oligonucleotides such as the phosphorothioates and 2′-alkoxy or 2′-alkoxyalkoxy derivatives, including 2′-O-methoxyethyl oligonucleotides (Martin, P., Helv. Chim. Acta 1995, 78, 486-504). It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotides.

The antisense compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids. APharmaceutically acceptable salts@ are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19).

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotides of the invention may additionally or alternatively be prepared to be delivered in a Aprodrug@ form. The term Aprodrug@ indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993.

For therapeutic or prophylactic treatment, oligonucleotides are administered in accordance with this invention. Oligonucleotide compounds of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the oligonucleotide. Such compositions and formulations are comprehended by the present invention.

Pharmaceutical compositions comprising the oligonucleotides of the present invention may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; El-Hariri et al., J. Pharm. Pharmacol. 1992 44, 651-654).

The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives.

Complex formulations comprising one or more penetration enhancers may be used. For example, bile salts may be used in combination with fatty acids to make complex formulations.

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)[Lee et al., Critical Reviews in Therapeutic Drug Carrier Systems 1991, page 92; Muranishi, Critical Reviews in Therapeutic Drug Carrier Systems 1990, 7, 1-33; Buur et al., J. Control Rel. 1990, 14, 43-51). Chelating agents have the added advantage of also serving as DNase inhibitors.

Surfactants include, for example, sodium lauryl sulfate, polyoxyethylene-9-lauryl ether and polyoxyethylene-20-cetyl ether (Lee et al., Critical Reviews in Therapeutic Drug Carrier Systems 1991, page 92); and perfluorochemical emulsions, such as FC-43 (Takahashi et al., J. Pharm. Phamacol. 1988, 40, 252-257).

Non-surfactants include, for example, unsaturated cyclic ureas, 1-alkyl- and 1-alkenylazacyclo-alkanone derivatives (Lee et al., Critical Reviews in Therapeutic Drug Carrier Systems 1991, page 92); and non-steroidal anti-inflammatory agents such as diclofenac sodium, indomethacin and phenylbutazone (Yamashita et al., J. Pharm. Pharmacol. 1987, 39, 621-626).

As used herein, “carrier compound” refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the oligonucleotides of the invention are introduced into a patient, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708).

The pharmaceutical compositions of the present invention may be administered in a number of ways depending upon whether local or systemic treatment is desired and upon the area to be treated. Administration may be topical (including ophthalmic, vaginal, rectal, intranasal, epidermal, and transdermal), oral or parenteral. Parenteral administration includes intravenous drip, subcutaneous, intraperitoneal or intramuscular injection, pulmonary administration, e.g., by inhalation or insufflation, or intracranial, e.g., intrathecal or intraventricular, administration. Oligonucleotides with at least one 2′-O-methoxyethyl modification are believed to be particularly useful for oral administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral administration may include sterile aqueous solutions which may also contain buffers, diluents and other suitable additives. In some cases it may be more effective to treat a patient with an oligonucleotide of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide).

For prophylactics and therapeutics, methods of preventing, inhibiting and treating allograft rejection are provided. The formulation of therapeutic compositions and their subsequent administration is believed to be within the skill in the art. While administration of therapeutics to the allograft recipient via varying routes prior to transplantation can serve to inhibit or prevent allograft rejection, prevention of allograft rejection ex vivo (perfusion of the allograft prior to transplantation) may be preferred. Methods of organ perfusion are well known in the art. In general, harvested tissues or organs (preferably heart or kidney) are perfused with the compositions of the invention in a pharmacologically acceptable carrier such as, for example, lactated Ringer's solution, University of Wisconsin (UW) solution, Euro-Collins solution or Sachs solution. Simple flushing of the organ or pulsatile perfusion may be used. Perfusion time is generally dependent on the length of ex vivo viability of the organ being transplanted; these viability times vary from organ to organ and are known in the art. Hearts and livers, for example, are generally transplanted within 4 to 6 hours of harvesting, whereas other organs may have longer ischemic viability. Kidneys, for example, may be transplanted up to 48 hours or even 72 hours after harvesting. Dosage may range from 0.001 μg to 500 g of oligonucleotide.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in vitro and in in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

The following examples illustrate the present invention and are not intended to limit the same.

### EXAMPLES

### Example 1

Synthesis of Oligonucleotides

Unmodified oligodeoxynucleotides are synthesized on an automated DNA synthesizer (Applied Biosystems model 380B) using standard phosphoramidite chemistry with oxidation by iodine. β-cyanoethyldiisopropyl-phosphoramidites are purchased from Applied Biosystems (Foster City, Calif.). For phosphorothioate oligonucleotides, the standard oxidation bottle was replaced by a 0.2 M solution of ³H-1,2-benzodithiole-3-one 1,1-dioxide in acetonitrile for the stepwise thiation of the phosphite linkages. The thiation cycle wait step was increased to 68 seconds and was followed by the capping step. Cytosines may be 5-methyl cytosines. (5-methyl deoxycytidine phosphoramidites available from Glen Research, Sterling, Va., or Amersham Pharmacia Biotech, Piscataway, N.J.)

2′-methoxy oligonucleotides are synthesized using 2′-methoxy β-cyanoethyldiisopropyl-phosphoramidites (Chemgenes, Needham, Mass.) and the standard cycle for unmodified oligonucleotides, except the wait step after pulse delivery of tetrazole and base is increased to 360 seconds. Other 2′-alkoxy oligonucleotides are synthesized by a modification of this method, using appropriate 2′-modified amidites such as those available from Glen Research, Inc., Sterling, Va.

2′-fluoro oligonucleotides are synthesized as described in Kawasaki et al. (J. Med. Chem. 1993, 36, 831-841). Briefly, the protected nucleoside N⁶-benzoyl-2′-deoxy-2′-fluoroadenosine is synthesized utilizing commercially available 9-β-D-arabinofuranosyladenine as starting material and by modifying literature procedures whereby the 2′-a-fluoro atom is introduced by a S_{N}2-displacement of a 2′-β-O-trifyl group. Thus N⁶-benzoyl-9-β-D-arabinofuranosyladenine is selectively protected in moderate yield as the 3′,5′-ditetrahydropyranyl (THP) intermediate. Deprotection of the THP and N⁶-benzoyl groups is accomplished using standard methodologies and standard methods are used to obtain the 5′-dimethoxytrityl- (DMT) and 5′-DMT-3′-phosphoramidite intermediates.

The synthesis of 2¹-deoxy-2′-fluoroguanosine is accomplished using tetraisopropyldisiloxanyl (TPDS) protected 9-β-D-arabinofuranosylguanine as starting material, and conversion to the intermediate diisobutyryl-arabinofuranosylguanosine. Deprotection of the TPDS group is followed by protection of the hydroxyl group with THP to give diisobutyryl di-THP protected arabinofuranosylguanine. Selective O-deacylation and triflation is followed by treatment of the crude product with fluoride, then deprotection of the THP groups. Standard methodologies are used to obtain the 5′-DMT- and 5′-DMT-3′-phosphoramidites.

Synthesis of 2′-deoxy-2′-fluorouridine is accomplished by the modification of a known procedure in which 2, 2′-anhydro-1-β-D-arabinofuranosyluracil is treated with 70% hydrogen fluoride-pyridine. Standard procedures are used to obtain the 5′-DMT and 5′-DMT-3′phosphoramidites.

2′-deoxy-2′-fluorocytidine is synthesized via amination of 2′-deoxy-2′-fluorouridine, followed by selective protection to give N⁴-benzoyl-2′-deoxy-2′-fluorocytidine. Standard procedures are used to obtain the 5′-DMT and 5′-DMT-3′phosphoramidites.

2′-(2-methoxyethyl)-modified amidites were synthesized according to Martin, P. (Helv. Chim. Acta 1995, 78, 486-506). For ease of synthesis, the last nucleotide may be a deoxynucleotide. 2-O—CH₂CH₂OCH₃cytosines may be 5-methyl cytosines.

Synthesis of 5-Methyl Cytosine Monomers:

2,2′-Anhydro[1-(β-D-arabinofuranosyl)-5-methyluridine]:

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 hours) to give a solid which was crushed to a light tan powder (57 g, 85% crude yield). The material was used as is for further reactions.

2′-O-Methoxyethyl-5-methyluridine:

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160?C. After heating for 48 hours at 155-160?C, the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product.

2′-O-Methoxyethyl-5′-O-dimethoxytrityl-5-methyluridine:

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/Hexane/Acetone (5:5:1) containing 0.5% Et₃NH. The pure fractions were evaporated to give 164 g of product. Approximately 20 g additional was obtained from the impure fractions to give a total yield of 183 g (57%).

3′-O-Acetyl-2′-O-methoxyethyl-5′-O-dimethoxytrityl-5-methyluridine:

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by tlc by first quenching the tlc sample with the addition of MeOH. Upon completion of the reaction, as judged by tlc, MeOH (50 mL) was added and the mixture evaporated at 35?C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/Hexane(4:1). Pure product fractions were evaporated to yield 96 g (84%).

3′-O-Acetyl-2′-O-methoxyethyl-5′-O-dimethoxytrityl-5-methyl-4-triazoleuridine:

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 hour using an overhead stirrer. POCl₃ was added dropwise, over a 30 minute period, to the stirred solution maintained at 0-10?C, and the resulting mixture stirred for an additional 2 hours. The first solution was added dropwise, over a 45 minute period, to the later solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

2′-O-Methoxyethyl-5′-O-dimethoxytrityl-5-methylcytidine:

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (tlc showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

N⁴-Benzoyl-2′-O-methoxyethyl-5′-O-dimethoxytrityl-5-methyl-cytidine:

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, tlc showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 9). The residue was chromatographed on a 1.5 kg silica column using EtOAc/Hexane (1:1) containing 0.5% Et₃NH as the eluting solvent. The pure product fractions were evaporated to give 90 g (90%) of the title compound.

N⁴-Benzoyl-2′-O-methoxyethyl-5′-O-dimethoxytrityl-5-methylcytidine-3′-amidite:

N⁴-Benzoyl-2′-O-methoxyethyl-5′-O-dimethoxytrityl-5-methylcytidine (74 g, 0.10 M) was dissolved in CH₂Cl₂ (1 L). Tetrazole diisopropylamine (7.1 g) and 2-cyanoethoxy-tetra(isopropyl)phosphite (40.5 mL, 0.123 M) were added with stirring, under a nitrogen atmosphere. The resulting mixture was stirred for 20 hours at room temperature (tlc showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAc\Hexane (3:1) as the eluting solvent. The pure fractions were combined to give 90.6 g (87%) of the title compound.

5-methyl-2′-deoxycytidine (5-me-C) containing oligonucleotides were synthesized according to published methods (Sanghvi et al., Nucl. Acids Res. 1993, 21, 3197-3203) using commercially available phosphoramidites (Glen Research, Sterling, Va., or ChemGenes, Needham, Mass.).

2′-O-(dimethylaminooxyethyl) nucleoside amidites:

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected witha benzoyl moiety in the case of adenosine and cytidine and with isobutyryl in the case of guanosine.

5′-O-tert-Butyldiphenylsilyl-O²-2′-anhydro-5-methyluridine:

O²-2-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 hours at ambient temperature. TLC (Rf 0.22, ethyl acetate) indicated a complete reaction. The solution was concentrated under reduced pressure to a thick oil. This was partitioned between dichloromethane (1 L) and saturated sodium bicarbonate (2×1 L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 hours) to 149 g (74.8%) of white solid. TLC and NMR were consistent with pure product.

5′-O-tert-Butyldiphenylsilyl-2′-O-(2-hydroxyethyl)-5-methyluridine:

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 hours (pressure <100 psig). The reaction vessel was cooled to ambient and opened. TLC (Rf 0.67 for desired product and Rf 0.82 for ara-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

2′-O-([2-phthalimidoxy)ethyl]-5′-t-butyldiphenylsilyl-5-methyluridine:

5′-O-tert-Butyldiphenylsilyl-2′-O-(2-hydroxyethyl)-5-methyluridine (20 g, 36.98 mol.) was mixed with triphenylphosphine (11.63 g, 44.36 mol.) and N-hydroxyphthalimide (7.24 g, 44.36 mol.). It was then dried over P₂O under high vacuum for two days at 40° C. The reaction mixture was flushed with argon and dry THF (369.8 mL, Aldrich, sure seal bottle) was added to get a clear solution. Diethyl-azodicarboxylate (6.98 mL, 44.36 mol.) was added dropwise to the reaction mixture. The rate of addition is maintained such that resulting deep red coloration is just discharged before adding the next drop. After the addition was complete, the reaction was stirred for 4 hours. By that time TLC showed the completion of the reaction (ethylacetate:hexane, 60:40). The solvent was evaporated in vacuum. Residue obtained was placed on a flash column and eluted with ethyl acetate:hexane (60:40), to get 2′-O-([2-phthalimidoxy)ethyl]-5′-t-butyldiphenylsilyl-5-methyluridine as white foam (21.819, 86%).

5′-O-tert-butyldiphenylsilyl-2′-O-[(2-formadoximinooxy)ethyl]-5-methyluridine:

2′-O-([2-phthalimidoxy)ethyl]-5′-t-butyldiphenylsilyl-5-methyluridine (3.1 g, 4.5 mol.) was dissolved in dry CH₂Cl₂ (4.5 mL) and methylhydrazine (300 mL, 4.64 mol.) was added dropwise at −10° C. to 0° C. After 1 hour the mixture was filtered, the filtrate was washed with ice cold CH₂Cl₂ and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eg.) was added and the mixture for 1 hour. Solvent was removed under vacuum; residue chromatographed to get 5′-O-tert-butyldiphenylsilyl-2′-O-[(2-formadoximinooxy) ethyl]-5-methyluridine as white foam (1.95, 78%).

5′-O-tert-Butyldiphenylsilyl-2′-O-[N,N-dimethylaminooxyethyl]-5-methyluridine:

5′-O-tert-butyldiphenylsilyl-2′-O-[(2-formadoximinooxy)ethyl]-5-methyluridine (1.77 g, 3.12 mol.) was dissolved in a solution of 1M pyridinium p-toluenesulfonate (PPTS) in dry MeOH (30.6 mL). Sodium cyanoborohydride (0.39 g, 6.13 mol.) was added to this solution at 10° C. under inert atmosphere. The reaction mixture was stirred for 10 minutes at 10° C. After that the reaction vessel was removed from the ice bath and stirred at room temperature for 2 hours, the reaction monitored by TLC (5% MeOH in CH₂Cl₂). Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mol.) was added and the reaction mixture was stirred at room temperature for 10 minutes. Reaction mixture cooled to 10° C. in an ice bath, sodium cyanoborohydride (0.39 g, 6.13 mol.) was added and reaction mixture stirred at 10° C. for 10 minutes. After 10 minutes, the reaction mixture was removed from the ice bath and stirred at room temperature for 2 hours. To the reaction mixture 5% NaHCO₃ (25 mL) solution was added and extracted with ethyl acetate (2×25 mL). Ethyl acetate layer was dried over anhydrous Na₂SO₄ and evaporated to dryness. The residue obtained was purified by flash column chromatography and eluted with 5% MeOH in CH₂Cl₂ to get 5′-O-tert-butyldiphenylsilyl-2′-O-[N,N-dimethylaminooxyethyl]-5-methyluridine as a white foam (14.6 g, 80%).

2′-O-(dimethylaminooxyethyl)-5-methyluridine:

Triethylamine trihydrofluoride (3.91 mL, 24.0 mol.) was dissolved in dry THF and triethylamine (1.67 mL, 12 mol., dry, kept over KOH). This mixture of triethylamine-2HF was then added to 5′-O-tert-butyldiphenylsilyl-2′-O-[N,N-dimethylaminooxyethyl]-5-methyluridine (1.40 g, 2.4 mol.) and stirred at room temperature for 24 hours. Reaction was monitored by TLC (5% MeOH in CH₂Cl₂). Solvent was removed under vacuum and the residue placed on a flash column and eluted with 10% MeOH in CH₂Cl₂ to get 2′-O-(dimethylaminooxyethyl)-5-methyluridine (766 mg, 92.5%).

5′-O-DMT-2′-O-(dimethylaminooxyethyl)-5-methyluridine:

2′-O-(dimethylaminooxyethyl)-5-methyluridine (750 mg, 2.17 mol.) was dried over P₂O₅ under high vacuum overnight at 40° C. It was then co-evaporated with anhydrous pyridine (20 mL). The residue obtained was dissolved in pyridine (11 mL) under argon atmosphere. 4-dimethylaminopyridine (26.5 mg, 2.60 mol.), 4,4′-dimethoxytrityl chloride (880 mg, 2.60 mol.) was added to the mixture and the reaction mixture was stirred at room temperature until all of the starting material disappeared. Pyridine was removed under vacuum and the residue chromatographed and eluted with 10% MeOH in CH₂Cl₂ (containing a few drops of pyridine) to get 5′-O-DMT-2′-O-(dimethylamino-oxyethyl)-5-methyluridine (1.13 g, 80%).

5′-O-DMT-2′-O-(2-N,N-dimethylaminooxyethyl)-5-methyluridine-3′-[(2-cyanoethyl)-N,N-diisopropylphosphoramidite]:

5′-O-DMT-2′-O-(dimethylaminooxyethyl)-5-methyluridine (1.08 g, 1.67 mol.) was co-evaporated with toluene (20 mL). To the residue N,N-diisopropylamine tetrazonide (0.29 g, 1.67 mol.) was added and dried over P₂O₅ under high vacuum overnight at 40° C. Then the reaction mixture was dissolved in anhydrous acetonitrile (8.4 mL) and 2-cyanoethyl-N,N,N¹,N¹-tetraisopropylphosphoramidite (2.12 mL, 6.08 mol.) was added. The reaction mixture was stirred at ambient temperature for 4 hours under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO₃ (40 mL). Ethyl acetate layer was dried over anhydrous Na₂SO₄ and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

Oligonucleotides having methylene (methylimino) (MMI) backbones are synthesized according to U.S. Pat. No. 5,378,825, which is coassigned to the assignee of the present invention and is incorporated herein in its entirety. For ease of synthesis, various nucleoside dimers containing MMI linkages are synthesized and incorporated into oligonucleotides. Other nitrogen-containing backbones are synthesized according to WO 92/20823 which is also coassigned to the assignee of the present invention and incorporated herein in its entirety.

Oligonucleotides having amide backbones are synthesized according to De Mesmaeker et al. (Acc. Chem. Res. 1995, 28, 366-374). The amide moiety is readily accessible by simple and well-known synthetic methods and is compatible with the conditions required for solid phase synthesis of oligonucleotides.

Oligonucleotides with morpholino backbones are synthesized according to U.S. Pat. No. 5,034,506 (Summerton and Weller).

Peptide-nucleic acid (PNA) oligomers are synthesized according to P. E. Nielsen et al. (Science 1991, 254, 1497-1500).

After cleavage from the controlled pore glass column (Applied Biosystems) and deblocking in concentrated ammonium hydroxide at 55° C. for 18 hours, the oligonucleotides are purified by precipitation twice out of 0.5 M NaCl with 2.5 volumes ethanol. Synthesized oligonucleotides were analyzed by polyacrylamide gel electrophoresis on denaturing gels or capillary gel electrophoresis and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al. (J. Biol. Chem. 1991, 266, 18162). Results obtained with HPLC-purified material were similar to those obtained with non-HPLC purified material.

### Example 2

Human Fas Oligonucleotide Sequences

Antisense oligonucleotides were designed to target human Fas. Target sequence data are from the APO-1 cDNA sequence published by Oehm, A., et al. (J. Biol. Chem., 1992, 267, 10709-10715); Genbank accession number X63717, provided herein as SEQ ID NO: 1. Oligonucleotides were synthesized as chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings.” The wings are composed of 2′-methoxyethyl (2′-MOE) nucleotides. The internucleoside (backbone) linkages are phosphorothioate (P═S) throughout the oligonucleotide. All 2′-MOE cytosines were 5-methyl-cytosines. These oligonucleotide sequences are shown in Table 1.

The C8161 melanoma cell line was obtained from Welch D. R., et al. (Int. J. Cancer, 1991, 47, 227-237). C8161 cells were cultured in RPMI 1640 medium plus 10% fetal bovine serum (Hyclone, Logan, Utah).

C8161 cells (5.5×10⁵ cells) were plated onto 100 cm plates. Two days later, the cells were washed once with OPTIMEM™ (Life Technologies, Rockville, Md.), then transfected with 300 nM oligonucleotide and 15 g/ml LIPOFECTIN⁷ (Life Technologies, Rockville, Md.), a 1:1 (w/w) liposome formulation of the cationic lipid N-[1-(2,3-dioleyloxy)propyl]-n,n,n-trimethylammonium chloride (DOTMA), and dioleoyl phosphotidylethanolamine (DOPE) in membrane filtered water. The cells were incubated with oligonucleotide for four hours, after which the media was replaced with fresh media and the cells incubated for another 20 hours.

Total cellular RNA was isolated using the RNEASY⁷ kit (Qiagen, Santa Clarita, Calif.). RNA was then separated on a 1% agarose gel, transferred to Hybond-N+membrane (Amersham, Arlington Heights, Ill.), a positively charged nylon membrane, and probed. A Fas probe was generated by random primer labeling of a RT-PCR amplified fragment of Fas mRNA.

A glyceraldehyde 3-phosphate dehydrogenase (G3PDH) probe was purchased from Clontech (Palo Alto, Calif.), Catalog Number 9805-1. The probes were labeled by random primer using the Large Fragment of DNA polymerase (Klenow fragment) (GIBCO BRL) as described in Maniatis, T., et al., Molecular Cloning: A Laboratory Manual, 1989. mRNA was quantitated by a PhosphoImager (Molecular Dynamics, Sunnyvale, Calif.).

Results of an initial screen of Fas antisense oligonucleotides is shown in Table 2. Oligonucleotides 17014 (SEQ ID NO. 5), 17015 (SEQ ID NO. 6), 17016 (SEQ ID NO. 7), 17017 (SEQ ID NO. 8), 17019 (SEQ ID NO. 10), 17020 (SEQ ID NO. 11), 17021 (SEQ ID NO. 12), 17022 (SEQ ID NO. 13), 17023 (SEQ ID NO. 14), 17024 (SEQ ID NO. 15), 17025 (SEQ ID NO. 16), 17026 (SEQ ID NO. 17), 17028 (SEQ ID NO. 19), 17029 (SEQ ID NO. 20), and 17030 (SEQ ID NO. 21) resulted in at least 60% inhibition of Fas mRNA expression in this assay. Oligonucleotides 17016 (SEQ ID NO. 7), 17017 (SEQ ID NO. 8), 17019 (SEQ ID NO. 10), 17020 (SEQ ID NO. 11), 17021 (SEQ ID NO. 12), 17022 (SEQ ID NO. 13), 17023 (SEQ ID NO. 14), 17024 (SEQ ID NO. 15), 17025 (SEQ ID NO. 16), and 17026 (SEQ ID NO. 17) resulted in at least 80% inhibition of Fas mRNA expression.

TABLE 1
Nucleotide Sequences of Human Fas Chimeric (deoxy gapped)
Phosphorothioate Oligonucleotides

| | NUCLEOTIDE SEQUENCE¹ | | TARGET GENE NUCLEOTIDE | GENE TARGET |
|---|---|---|---|---|
| ISIS NO. | (5′ −> 3′) | SEQ ID NO: | CO-ORDINATES² | REGION |
| 17012 | CGTAAACCGCTTCCCTCACT | 3 | 0040-0059 | 5′-UTR |
| 17013 | GTGTTCCGTGCCAGTGCCCG | 4 | 0085-0104 | 5′-UTR |
| 17014 | GCCCAGCATGGTTGTTGAGC | 5 | 0210-0229 | AUG |
| 17015 | CTTCCTCAATTCCAATCCCT | 6 | 0318-0337 | coding |
| 17016 | CTTCTTGGCAGGGCACGCAG | 7 | 0463-0482 | coding |
| 17017 | TGCACTTGGTATTCTGGGTC | 8 | 0583-0602 | coding |
| 17018 | GCTGGTGAGTGTGCATTCCT | 9 | 0684-0703 | coding |
| 17019 | CATTGACACCATTCTTTCGA | 10 | 0967-0986 | coding |
| 17020 | TCACTCTAGACCAAGCTTTG | 11 | 1214-1233 | stop |
| 17021 | CCCAGTAAAAAACCAAGCAG | 12 | 1305-1324 | 3′-UTR |
| 17022 | TATGTTGGCTCTTCAGCGCT | 13 | 1343-1362 | 3′-UTR |
| 17023 | ATTTGGGTACTTAGCATGCC | 14 | 1452-1471 | 3′-UTR |
| 17024 | GGGTTAGCCTGTGGATAGAC | 15 | 1568-1587 | 3′-UTR |
| 17025 | CAAAGTGGCCTGCCTGTTCA | 16 | 1641-1660 | 3′-UTR |
| 17026 | TTGAGCCAGTAAAATGCATA | 17 | 1890-1909 | 3′-UTR |
| 17027 | TGAGCACCAAGGCAAAAATG | 18 | 1983-2002 | 3′-UTR |
| 17028 | TCTTGCCTTTTGGTGGACTA | 19 | 2057-2076 | 3′-UTR |
| 17029 | AGCAGGTTTTACATGGGACA | 20 | 2222-2241 | 3′-UTR |
| 17030 | GGTATGACAAGAGCAATTCC | 21 | 2291-2310 | 3′-UTR |
| 17031 | GGTGGTTCCAGGTATCTGCT | 22 | 2450-2469 | 3′-UTR |
| 17032 | TATAATTCCAAACACAAGGG | 23 | 2503-2522 | 3′-UTR |

¹Emboldened residues are 2′-methoxyethoxy residues, 2′-methoxyethoxy cytosine residues are 5-methyl-cytosines; all linkages are phosphorothioate linkages.
²Coordinates from Genbank Accession No. X63717, locus name “HSAPO1”, SEQ ID NO. 1.

Emboldened residues are 2′-methoxyethoxy residues, 2′-methoxyethoxy cytosine residues are 5-methyl-cytosines; all linkages are phosphorothioate linkages. 2 Coordinates from Genbank Accession No. X63717, locus name “HSAPO1”, SEQ ID NO. 1.

TABLE 2
Inhibition of Human Fas mRNA expression in C8161 Cells by
Chimeric (deoxy gapped) Phosphorothioate Oligonucleotides

| | SEQ | GENE | | |
|---|---|---|---|---|
| ISIS | ID | TARGET | % mRNA | % mRNA |
| No: | NO: | REGION | EXPRESSION | INHIBITION |
| control | — | — | 100.0% | 0.0% |
| 17012 | 3 | 5′-UTR | 98.7% | 1.3% |
| 17013 | 4 | 5′-UTR | 81.3% | 18.7% |
| 17014 | 5 | AUG | 27.1% | 72.9% |
| 17015 | 6 | coding | 30.0% | 70.0% |
| 17016 | 7 | coding | 8.7% | 91.3% |
| 17017 | 8 | coding | 10.1% | 89.9% |
| 17018 | 9 | coding | 186.1% | — |
| 17019 | 10 | coding | 12.9% | 87.1% |
| 17020 | 11 | stop | 7.3% | 92.7% |
| 17021 | 12 | 3′-UTR | 15.8% | 84.2% |
| 17022 | 13 | 3′-UTR | 15.1% | 84.9% |
| 17023 | 14 | 3′-UTR | 11.4% | 88.6% |
| 17024 | 15 | 3′-UTR | 11.3% | 88.7% |
| 17025 | 16 | 3′-UTR | 9.4% | 90.6% |
| 17026 | 17 | 3′-UTR | 19.6% | 80.4% |
| 17027 | 18 | 3′-UTR | 54.3% | 45.7% |
| 17028 | 19 | 3′-UTR | 26.6% | 73.4% |
| 17029 | 20 | 3′-UTR | 23.6% | 76.4% |
| 17030 | 21 | 3′-UTR | 35.5% | 64.5% |
| 17031 | 22 | 3′-UTR | 75.1% | 24.9% |
| 17032 | 23 | 3′-UTR | 58.4% | 41.6% |

The most active oligonucleotide, 17020 (SEQ ID NO. 11) was used in a dose response experiment. C8161 cells were grown and treated as described above except the concentration was varied as shown in Table 3. The LIPOFECTIN⁷ to oligonucleotide ratio was maintained at 3 ?g/ml LIPOFECTIN⁷ per 100 nM oligonucleotide. RNA was isolated and quantitated as described above. Included in this experiment were control oligonucleotides with 2, 4, or 6 base mismatches or a scrambled control oligonucleotide. These controls were tested at 3OOnM.

Results are shown in Table 3.

TABLE 3
Dose Response of C8161 cells to ISIS 17020

| | SEQ ID | ASO Gene | | % RNA | % RNA |
|---|---|---|---|---|---|
| ISIS # | NO: | Target | Dose | Expression | Inhibition |
| control | — | — | — | 100% | — |
| 17020 | 11 | stop | 25 nM | 50.6% | 49.4% |
| ″ | ″ | ″ | 50 nM | 44.9% | 55.1% |
| ″ | ″ | ″ | 100 nM | 28.1% | 71.9% |
| ″ | ″ | ″ | 150 nM | 21.8% | 78.2% |
| ″ | ″ | ″ | 200 nM | 24.2% | 75.8% |
| ″ | ″ | ″ | 300 nM | 19.3% | 80.7% |
| ″ | ″ | ″ | 400 nM | 20.6% | 79.4% |

From the dose response curve, oligonucleotide 17020 has an IC₅₀ of about 25 nM. Control oligonucleotides with 2, 4, or 6 base mismatches or a scrambled control oligonucleotide showed no inhibition of Fas mRNA expression.

### Example 3

Human FasL Oligonucleotide Sequences Antisense oligonucleotides were designed to target human FasL. Target sequence data are from the Fas ligand cDNA sequence published by Mita, E. et al. (Biochem. Biophys. Res. Commun., 1994, 204, 468-474); Genbank accession number D38122, provided herein as SEQ ID NO: 24. Oligonucleotides were synthesized as chimeric oligonucleotides (“gapmers”) 20 nucleotides in length, composed of a central “gap” region consisting of ten 2'deoxynucleotides, which is flanked on both sides (5′ and 3′ directions) by five nucleotide “wings.” The wings are composed of 2′methoxyethyl (2′MOE) nucleotides. The internucleoside (backbone) linkages are phosphorothioate (P═S) throughout the oligonucleotide. All 2′-MOE cytosines were 5-methyl-cytosines. These oligonucleotide sequences are shown in Table 4.

NHEK cells, a human epidermal keratinocyte cell line was obtained from Clonetics (Walkersville, Md.). NHEK were grown in Keratinocyte Growth Media (KGM) (Gibco BRL, Gaithersburg, Md.) containing 5 NG/ml of EG., bovine pituitary extract. NHEK cells were used at passage 6.

NHEK were grown to 60-80% confluency, washed once with basal media, and then incubated for 4 hours with 5 ml of basal media containing 10 ?g/ml LIPOFECTIN⁷ (Gibco BRL, Gaithersburg, Md.) and 300 nM of oligonucleotide. The media was replaced with fresh media and cells were incubated for an additional 20 hours.

Total cellular RNA was isolated by guanidinium isothiocyante extraction followed by ultracentrifugation (see Ausubel, F. M. et al., Current Protocols in Molecular Biology, 1993, John Wiley & Sons, Inc.). Northern blotting was performed as described in Example 2. A FasL probe was generated by PCR using FasL primers (Life Technologies). Signals from Northern blots were quantitated as described in Example 2.

Results are shown in Table 5. Oligonucleotides 16171 (SEQ ID NO. 36), 16172 (SEQ ID NO. 37), 16178 (SEQ ID NO. 43) and 16179 (SEQ ID NO. 44) resulted in at least 45% inhibition of Fas ligand mRNA expression in this assay.

TABLE 4
Nucleotide Sequences of Human FasL Chimeric (deoxy gapped)
Phosphorothioate Oligonucleotides

| | NUCLEOTIDE SEQUENCE¹ | | TARGET GENE NUCLEOTIDE | GENE TARGET |
|---|---|---|---|---|
| ISIS NO. | (5′ −> 3′) | SEQ ID NO: | CO-ORDINATES² | REGION |
| 16161 | CCATAGCTAAGGGAAACACC | 26 | 0034-0053 | 5′-UTR |
| 16162 | GCCAGCCCCAGCAAACGGTT | 27 | 0152-0171 | 5′-UTR |
| 16163 | TGCATGGCAGCTGGTGAGTC | 28 | 0174-0193 | AUG |
| 16164 | GGAAGAACTGTGCCTGGAGG | 29 | 0261-0280 | coding |
| 16165 | TGGCAGCGGTAGTGGAGGCA | 30 | 0376-0395 | coding |
| 16166 | GCTGTGTGCATCTGGCTGGT | 31 | 0540-0559 | coding |
| 16167 | AATGGGCCACTTTCCTCAGC | 32 | 0614-0633 | coding |
| 16168 | GCAGGTTGTTGCAAGATTGA | 33 | 0785-0804 | coding |
| 16169 | AAGATTGAACACTGCCCCCA | 34 | 0922-0941 | coding |
| 16170 | AATCCCAAAGTGCTTCTCTT | 35 | 1033-1052 | stop |
| 16171 | TTCTCGGTGCCTGTAACAAA | 36 | 1069-1088 | 3′-UTR |
| 16172 | GCTACAGACATTTTGAACCC | 37 | 1169-1188 | 3′-UTR |
| 16173 | CCGTCATATTCCTCCATTTG | 38 | 1220-1239 | 3′-UTR |
| 16174 | CCCTCTTCACATGGCAGCCC | 39 | 1256-1275 | 3′-UTR |
| 16175 | GGTGTCCTTTTCAATCTGCC | 40 | 1338-1357 | 3′-UTR |
| 16176 | CAGTCCCCCTTGAGGTAGCA | 41 | 1385-1404 | 3′-UTR |
| 16177 | GTGAAGATGCTGCCAGTGGG | 42 | 1503-1522 | 3′-UTR |
| 16178 | CCCCTACAATTGGCACTGGA | 43 | 1618-1637 | 3′-UTR |
| 16179 | TCTTGACCAAATGCAACCCA | 44 | 1714-1733 | 3′-UTR |

¹Emboldened residues are 2′-methoxyethoxy residues, 2′-methoxyethoxy cytosine residues are 5-methyl-cytosines; all linkages are phosphorothioate linkages.
²Coordinates from Genbank Accession No. D31822, locus name “HUMHPC”, SEQ ID NO. 24.

TABLE 5
Inhibition of Human FasL mRNA expression in NHEK Cells by
Chimeric (deoxy gapped) Phosphorothioate Oligonucleotides

| | SEQ | GENE | | |
|---|---|---|---|---|
| ISIS | ID | TARGET | % mRNA | % mRNA |
| No: | NO: | REGION | EXPRESSION | INHIBITION |
| control | — | — | 100.0% | 0.0% |
| 16161 | 26 | 5′-UTR | 127.0% | — |
| 16162 | 27 | 5′-UTR | 136.0% | — |
| 16163 | 28 | AUG | 119.0% | — |
| 16164 | 29 | coding | 110.0% | — |
| 16165 | 30 | coding | 124.0% | — |
| 16166 | 31 | coding | 131.0% | — |
| 16167 | 32 | coding | 142.0% | — |
| 16168 | 33 | coding | 137.0% | — |
| 16169 | 34 | coding | 111.0% | — |
| 16170 | 35 | stop | 108.0% | — |
| 16171 | 36 | 3′-UTR | 53.0% | 47.0% |
| 16172 | 37 | 3-UTR | 50.0% | 50.0% |
| 16173 | 38 | 3′-UTR | 91.0% | 9.0% |
| 16174 | 39 | 3′-UTR | 136.0% | — |
| 16175 | 40 | 3′-UTR | 69.0% | 31.0% |
| 16176 | 41 | 3′-UTR | 130.0% | — |
| 16177 | 42 | 3′-UTR | 94.0% | 6.0% |
| 16178 | 43 | 3′-UTR | 55.0% | 45.0% |
| 16179 | 44 | 3′-UTR | 48.0% | 52.0% |

### Example 4

Human Fap-1 Oligonucleotide Sequences

Antisense oligonucleotides were designed to target human Fap-1. Target sequence data are from the protein tyrosine phosphatase (PTP-BAS, type 1) CDNA sequence published by Maekawa, K. et al. (FEBS Lett., 1994, 337, 200-206); Genbank accession number D21209, provided herein as SEQ ID NO: 45. Oligonucleotides were synthesized as chimeric oligonucleotides (“gapmers”) 20 nucleotides in length, composed of a central “gap” region consisting of ten 2'deoxynucleotides, which is flanked on both sides (5′ and 3′ directions) by five nucleotide “wings.” The wings are composed of 2′methoxyethyl (2′MOE) nucleotides. The internucleoside (backbone) linkages are phosphorothioate (P═S) throughout the oligonucleotide. All 2′-MOE cytosines and 2′-deoxy cytosines were 5-methyl-cytosines. These oligonucleotide sequences are shown in Table 6.

C8161 cells were grown and treated with oligonucleotide as described in Example 2 except that 9 ?g/ml LIPOFECTIN⁷ was used. mRNA was isolated and quantitated as described in Example 2. Results are shown in Table 7. Oligonucleotides 16148 (SEQ ID NO. 48), 18470 (SEQ ID NO. 50), 18471 (SEQ ID NO. 51), 18472 (SEQ ID NO. 52), 18473 (SEQ ID NO. 53), 18479 (SEQ ID NO. 58), 18480 (SEQ ID NO. 59), 18481 (SEQ ID NO. 60), and 18485 (SEQ ID NO. 64) resulted in greater than 60% inhibition of Fap-1 mRNA expression in this assay. Oligonucleotide 18479 (SEQ ID NO. 58) resulted in greater than 85% inhibition.

TABLE 6
Nucleotide Sequences of Human FAP-1 Chimeric (deoxy gapped)
Phosphorothioate Oligonucleotides

| | NUCLEOTIDE SEQUENCE¹ | | TARGET GENE NUCLEOTIDE | GENE TARGET |
|---|---|---|---|---|
| ISIS NO. | (5′ −> 3′) | SEQ ID NO: | CO-ORDINATES² | REGION |
| 18467 | ACGTGCATATTACCGGCTGG | 47 | 0052-0071 | AUG |
| 18468 | GAGAAATGATGAAGCCAAGG | 48 | 0201-0220 | coding |
| 18469 | GTTGGCTCTGAGGCACTTCA | 49 | 0405-0424 | coding |
| 18470 | TTTGTCTCTCTCGGATTCGG | 50 | 1200-1219 | coding |
| 18471 | GCCAAAGAAATTCCTCAGTT | 51 | 1664-1683 | coding |
| 18472 | AAGGATGCCAGCAATAAGGA | 52 | 2158-2177 | coding |
| 18473 | GGTCTTCAATGGATGAGGAG | 53 | 3189-3208 | coding |
| 18474 | GTGGTGATCCTTGGAAGAAG | 54 | 3701-3720 | coding |
| 18475 | TCCACTCCCACTGCTGTCAC | 55 | 5021-5040 | coding |
| 18476 | TTCTCTGATTGCCTTTGGTT | 56 | 5472-5491 | coding |
| 18478 | GCAACTCATCATTTCCCCAT | 57 | 6513-6532 | coding |
| 18479 | CCAGAGGCTCTTTTCATGTC | 58 | 7520-7539 | stop |
| 18480 | GCATCCAGAGGCTCTTTTCA | 59 | 7524-7543 | 3′-UTR |
| 18481 | GCTGGAGGTTAAGGAGAGAA | 60 | 7552-7571 | 3′-UTR |
| 18482 | TTTGGATAGAGAGCAGGAGT | 61 | 7574-7593 | 3′-UTR |
| 18483 | TTTCAAGAAGAATACCCCTA | 62 | 7648-7667 | 3′-UTR |
| 18484 | GCTGCCTTTAATCATCCCTA | 63 | 7760-7779 | 3′-UTR |
| 18485 | ACTGGTTTCAAGTATCCCCT | 64 | 7891-7910 | 3′-UTR |

¹Emboldened residues are 2′-methoxyethoxy residues, 2′-methoxyethoxy cytosine residues and 2′-OH cytosine residues are 5-methyl-cytosines; all linkages are phosphorothioate linkages.
²Coordinates from Genbank Accession No. D21209, locus name “HUMPTPB1”, SEQ ID NO. 45.

TABLE 7
Inhibition of Human Fap-1 mRNA expression in C8161 Cells by
Chimeric (deoxy gapped) Phosphorothioate Oligonucleotides

| | SEQ | GENE | | |
|---|---|---|---|---|
| ISIS | ID | TARGET | % mRNA | % mRNA |
| No: | NO: | REGION | EXPRESSION | INHIBITION |
| control | — | — | 100.0% | 0.0% |
| 18468 | 48 | coding | 33.4% | 66.6% |
| 18469 | 49 | coding | 71.9% | 28.1% |
| 18470 | 50 | coding | 33.2% | 66.8% |
| 18471 | 51 | coding | 33.3% | 66.7% |
| 18472 | 52 | coding | 26.9% | 73.1% |
| 18473 | 53 | coding | 28.3% | 71.7% |
| 18474 | 54 | coding | 51.9% | 48.1% |
| 18475 | 55 | coding | 46.2% | 53.8% |
| 18476 | 56 | coding | 133.6% | — |
| 18479 | 58 | stop | 11.6% | 88.4% |
| 18480 | 59 | 3′-UTR | 30.8% | 69.2% |
| 18481 | 60 | 3′-UTR | 35.2% | 64.8% |
| 18482 | 61 | 3′-UTR | 55.0% | 45.0% |
| 18483 | 62 | 3′-UTR | 55.3% | 44.7% |
| 18485 | 64 | 3′-UTR | 35.6% | 64.4% |

### Example 5

Mouse Fas Oligonucleotide Sequences

Antisense oligonucleotides were designed to target mouse fas. Target sequence data are from the Fas cDNA sequence published by Watanabe-Fukunaga, R. et al. (J. Immunol., 1992, 148, 1274-1297); Genbank accession number M83649, provided herein as SEQ ID NO: 65. Oligonucleotides were synthesized as chimeric oligonucleotides (“gapmers”) 20 nucleotides in length, composed of a central “gap” region consisting of ten 2'deoxynucleotides, which is flanked on both sides (5′ and 3′ directions) by five nucleotide “wings.” The wings are composed of 2′methoxyethyl (2′MOE) nucleotides. The internucleoside (backbone) linkages are phosphorothioate (P═S) throughout the oligonucleotide. All 2′-MOE and 2′-OH cytosines were 5-methyl-cytosines. Oligonucleotide sequences are shown in Table 8.

AML12 cells, a murine hepatocyte cell line, was obtained from ATCC (Manassas, Va.). AML12 cells were cultured in a 1:1 mixture of DMEM and F12 medium with 5.0 ?g/ml insulin, 5.0 ?g/ml transferrin, 5.0 NG/ml selenium, 0.04 ?g/ml dexamethasone and 10% FBS (all cell culture reagents available from Life Technologies).

AML12 cells were transfected with oligonucleotides as described in Example 2 for C8161 cells except oligonucleotide treatment was for six hours. For an initial screen, AML12 cells were transfected with 300 nM oligonucleotide and RNA collected 24 hours later.

Total cellular RNA was isolated using the RNEASY⁷ kit (Qiagen, Santa Clarita, Calif.). RNAse protection experiments were conducted using RIBOQUAN™ kits and the mAPO-2 Custom Probe Set set according to the manufacturer's instructions (Pharmingen, San Diego, Calif.). mRNA levels were quantitated using a PhosphorImager (Molecular Dynamics, Sunnyvale, Calif.).

Results are shown in Table 9. Oligonucleotides 22017 (SEQ ID NO. 67), 22018 (SEQ ID NO. 68), 22019 (SEQ ID NO. 69), 22023 (SEQ ID NO. 73), 22024 (SEQ ID NO. 74), 22025 (SEQ ID NO. 75), 22026 (SEQ ID NO. 76), 22027 (SEQ ID NO. 77), 22028 (SEQ ID NO. 78), 22030 (SEQ ID NO. 80) and 22032 (SEQ ID NO. 82) gave better than 40% inhibition of Fas mRNA in this assay. Oligonucleotides 22018 (SEQ ID NO. 68), 22023 (SEQ ID NO. 73), 22026 (SEQ ID NO. 76), 22028 (SEQ ID NO. 78), and 22030 (SEQ ID NO. 80) gave better than 60% inhibition of Fas mRNA.

TABLE 8
Nucleotide Sequences of Mouse Fas Chimeric (deoxy gapped)
Phosphorothioate Oligonucleotides

| | NUCLEOTIDE SEQUENCE¹ | | TARGET GENE NUCLEOTIDE | GENE TARGET |
|---|---|---|---|---|
| ISIS NO. | (5′ −> 3′) | SEQ ID NO: | CO-ORDINATES² | REGION |
| 22017 | GCAGCAAGGGAAAACAGCGG | 67 | 0026-0045 | 5′-UTR |
| 22018 | CCACAGCATGTCTGCAGCAA | 68 | 0039-0058 | AUG |
| 22019 | TTTCATGAACCCGCCTCCTC | 69 | 0148-0167 | coding |
| 22020 | GGGTCAGGGTGCAGTTTGTT | 70 | 0385-0404 | coding |
| 22021 | GAGGCGCAGCGAACACAGTG | 71 | 0461-0480 | coding |
| 22022 | CATAGGCGATTTCTGGGACT | 72 | 0542-0561 | coding |
| 22023 | TCCAGCACTTTCTTTTCCGG | 73 | 0616-0635 | coding |
| 22024 | GGTTTCACGACTGGAGGTTC | 74 | 0663-0682 | coding |
| 22025 | CTTCAGCAATTCTCGGGATG | 75 | 0721-0740 | coding |
| 22026 | GCCCTCCTTGATGTTATTTT | 76 | 0777-0796 | coding |
| 22027 | GGTACCAGCACAGGAGCAGC | 77 | 0853-0872 | coding |
| 22028 | CGGCTTTTTTGAGACCCTTG | 78 | 0910-0929 | coding |
| 22029 | GTGTCTGGGGTTGATTTTCC | 79 | 0980-0999 | coding |
| 22030 | TCTCCTCTCTTCATGGCTGG | 80 | 1048-1067 | 3′-UTR |
| 22031 | GGCATTCATTTTGTTTCCAT | 81 | 1084-1103 | 3′-UTR |
| 22032 | TCCCTGGAACCTGCTAGTCA | 82 | 1180-1199 | 3′-UTR |
| 22033 | TCAGCAACTGCAGAGAATAA | 83 | 1209-1228 | 3′-UTR |
| 22034 | GCAGATTCCACTTCACATTT | 84 | 1290-1309 | 3′-UTR |
| 22035 | AAGGTCTTCAATTAACTGCG | 85 | 1372-1391 | 3′-UTR |

¹Emboldened residues are 2′-methoxyethoxy residues, 2′-methoxyethoxy cytosine residues and 2′-OH cytosine residues are 5-methyl-cytosines; all linkages are phosphorothioate linkages.
²Coordinates from Genbank Accession No. M83649, locus name “MUSFASANT”, SEQ ID NO. 65.

TABLE 9
Inhibition of Mouse Fas mRNA expression in AML12 Cells by
Chimeric (deoxy gapped) Phosphorothioate Oligonucleotides

| | SEQ | GENE | | |
|---|---|---|---|---|
| ISIS | ID | TARGET | % mRNA | % mRNA |
| No: | NO: | REGION | EXPRESSION | INHIBITION |
| control | — | — | 100% | 0% |
| LIPOFECTIN⁷ | — | — | 136% | — |
| 22017 | 67 | 5′-UTR | 44% | 56% |
| 22018 | 68 | AUG | 38% | 62% |
| 22019 | 69 | coding | 56% | 44% |
| 22020 | 70 | coding | 69% | 31% |
| 22021 | 71 | coding | 77% | 23% |
| 22022 | 72 | coding | 77% | 23% |
| 22023 | 73 | coding | 37% | 63% |
| 22024 | 74 | coding | 49% | 51% |
| 22025 | 75 | coding | 57% | 43% |
| 22026 | 76 | coding | 31% | 69% |
| 22027 | 77 | coding | 53% | 47% |
| 22028 | 78 | coding | 28% | 72% |
| 22029 | 79 | coding | 82% | 18% |
| 22030 | 80 | 3′-UTR | 22% | 78% |
| 22031 | 81 | 3′-UTR | 76% | 24% |
| 22032 | 82 | 3′-UTR | 47% | 53% |
| 22033 | 83 | 3′-UTR | 103% | — |
| 22034 | 84 | 3′-UTR | 80% | 20% |
| 22035 | 85 | 3′-UTR | 98% | 2% |

### Example 6

Dose Response of Antisense Chimeric (Deoxy Gapped) Phosphorothioate Oligonucleotide Effects on Mouse Fas mRNA Levels in AML12 Cells

Oligonucleotides 22019 (SEQ ID. NO. 69), 22023 (SEQ ID. NO. 73) and 22028 (SEQ ID. NO. 78) was chosen for a dose response study. AML12 cells were grown, treated and processed as described in Example 5.

Results are shown in Table 10. IC₅₀s were 150 nM or less and maximal inhibition seen was greater than 80%.

TABLE 10
Dose Response of AML12 cells to Fas
Chimeric (deoxy gapped) Phosphorothioate Oligonucleotides

| | SEQ ID | ASO Gene | | % mRNA | % mRNA |
|---|---|---|---|---|---|
| ISIS # | NO: | Target | Dose | Expression | Inhibition |
| control | — | — | — | 100% | — |
| 22019 | 69 | coding | 75 nM | 60% | 40% |
| ″ | ″ | ″ | 150 nM | 53% | 47% |
| ″ | ″ | ″ | 300 nM | 34% | 66% |
| ″ | ″ | ″ | 500 nM | 14% | 86% |
| 22023 | 73 | coding | 75 nM | 61% | 39% |
| ″ | ″ | ″ | 150 nM | 28% | 72% |
| ″ | ″ | ″ | 300 nM | 22% | 78% |
| ″ | ″ | ″ | 500 nM | 20% | 80% |
| 22028 | 78 | coding | 75 nM | 57% | 43% |
| ″ | ″ | ″ | 150 nM | 49% | 51% |
| ″ | ″ | ″ | 300 nM | 42% | 58% |
| ″ | ″ | ″ | 500 nM | 45% | 55% |

A similar experiment was performed which included mismatch control oligonucleotides (2, 4, 6 or 8 base mismatches). None of these control oligonucleotides inhibited Fas mRNA expression.

### Example 7

Inhibition of Fas Expression in Balb/c Mice by Fas Antisense Chimeric (Deoxy Gapped) Phosphorothioate Oligonucleotides

Balb/c mice were used to assess the activity of Fas antisense oligonucleotides. Female Balb/c mice, 8 to 10 weeks old, were intra peritoneally injected with oligonucleotide at 100 mg/kg mouse body weight. Mice were injected daily for four days. Control mice were injected with a saline solution. After the fourth day, the livers were removed from the animals and analyzed for Fas mRNA expression. RNA was extracted using the RNEASY⁷ kit (Qiagen, Santa Clarita, Calif.) and quantitated using RPA as described in Example 5.

Results are shown in Table 11. Maximal inhibition seen in this assay was 80%.

TABLE 11
Inhibition of Mouse Fas mRNA expression in Balb/c Mice by
Chimeric (deoxy gapped) Phosphorothioate Oligonucleotides

| | SEQ | GENE | | |
|---|---|---|---|---|
| ISIS | ID | TARGET | % mRNA | % mRNA |
| No: | NO: | REGION | EXPRESSION | INHIBITION |
| control | — | — | 100% | 0% |
| 22019 | 69 | coding | 40% | 60% |
| 22023 | 73 | coding | 20% | 80% |
| 22028 | 78 | coding | 21% | 79% |

A dose response experiment was performed in Balb/c mice using oligonucleotides 22023 (SEQ ID NO. 73) and 22028 (SEQ ID NO. 78). Mice were treated as described above except the concentration of oligonucleotide was varied as shown in Table 12. Results are shown in Table 12. IC₅₀s for these oligonucleotides is estimated to be about 9 mg/kg. Maximal inhibition seen was greater than 90%.

TABLE 12
Dose Response of Balb/c to Fas
Chimeric (deoxy gapped) Phosphorothioate Oligonucleotides

| | SEQ ID | ASO Gene | | % mRNA | % mRNA |
|---|---|---|---|---|---|
| ISIS # | NO: | Target | Dose | Expression | Inhibition |
| control | — | — | — | 100% | — |
| 22023 | 73 | coding | 6 mg/kg | 66% | 34% |
| ″ | ″ | ″ | 12 mg/kg | 40% | 60% |
| ″ | ″ | ″ | 25 mg/kg | 26% | 74% |
| ″ | ″ | ″ | 50 mg/kg | 8% | 92% |
| ″ | ″ | ″ | 100 | 6% | 94% |
| | | | mg/kg | | |
| 22028 | 78 | coding | 6 mg/kg | 65% | 35% |
| ″ | ″ | ″ | 12 mg/kg | 40% | 60% |
| ″ | ″ | ″ | 25 mg/kg | 17% | 83% |
| ″ | ″ | ″ | 50 mg/kg | 12% | 88% |
| ″ | ″ | ″ | 100 | 13% | 87% |
| | | | mg/kg | | |

Oligonucleotide 22023 (SEQ ID NO. 73) was chosen for a time course study. Balb/c mice were treated as described above except that doses of 6 mg/kg and 12 mg/kg were used and treatment time (in days) was varied as shown in Table 13.

Results are shown in Table 13. Increasing the treatment time, in general, gave better results.

TABLE 13
Time Course of Balb/c to Fas Chimeric (deoxy gapped)
Phosphorothioate Oligonucleotide

| | SEQ ID | ASO Gene | | Treatment | % mRNA |
|---|---|---|---|---|---|
| ISIS # | NO: | Target | Dose | Time | Inhibition |
| control | — | — | — | — | — |
| 22023 | 73 | coding | 6 mg/kg | 2 d | 54% |
| ″ | ″ | ″ | ″ | 4 d | 55% |
| ″ | ″ | ″ | ″ | 7 d | 84% |
| ″ | ″ | ″ | ″ | 12 d | 87% |
| 22023 | 73 | coding | 12 mg/kg | 2 d | 40% |
| ″ | ″ | ″ | ″ | 4 d | 79% |
| ″ | ″ | ″ | ″ | 7 d | 92% |
| ″ | ″ | ″ | ″ | 12 d | 82% |

The effect of oligonucleotides 22023 (SEQ ID NO. 69) and 22028 (SEQ ID NO. 78) on Fas protein expression was examined. Balb/c mice were injected with oligonucleotide as described above. Lpr mice (Jackson Laboratory, Bar Harbor, ME), a Fas knockout strain, were used as a control. Four hours after the last dose, the mice were sacrificed and a piece of liver was frozen in O.C.T. compound (Sakura Finetek USA, Inc., Torrance, Calif.). The liver was fixed for 1 minute in acetone, then stained with Fas antibody (rabbit anti rat/mouse fas, Research Diagnostics, Inc., Flanders, NJ) at 0.7 μg/ml for 45 minutes. A second antibody (HRP conjugated donkey anti-rabbit, Jackson Laboratory) was then added at 1:100 dilution for 30 minutes. Then DAB (DAKO Corporation, Carpinteria, Calif.) was added for color development. Tissue sections were visualized under a microscope.

Treatment with Fas antisense oligonucleotides reduced Fas protein expression to levels similar to those in Lpr mice.

### Example 8

Effect of Fas Antisense Oligonucleotides in a Con A Murine Model for Hepatitis

Concanavalin A-induced hepatitis is used as a murine model for autoimmune hepatitis (Mizuhara, H., et al., J. Exp. Med., 1994, 179, 1529-1537). It has been shown that this type of liver injury is mediated by Fas (Seino, K., et al., Gastroenterology 1997, 113, 1315-1322). Certain types of viral hepatitis, including Hepatitis C, are also mediated by Fas (J. Gastroenterology and Hepatology, 1997, 12, S223-S226). Female Balb/c between the ages of 6 weeks and 3 months were used to assess the activity of Fas antisense oligonucleotides. For determining the effect of Fas antisense oligonucleotides on Fas mRNA expression, mice were injected intra peritoneally with oligonucleotide 22023 (SEQ ID NO. 73) at 50 mg/kg or 100 mg/kg, daily for 4 days. The pretreated mice were then intravenously injected with 0.3 mg concanavalin A (Con A) to induce liver injury. Within 24 hours following Con A injection, the livers were removed from the animals and RNA isolated using the RNEASY⁷ kit (Qiagen, Santa Clarita, Calif.) and quantitated using RPA as described in Example 5.

Results are shown in Table 14.

TABLE 14
Reduction of Balb/c Liver Fas mRNA with Fas Antisense
Chimeric (deoxy gapped) Phosphorothioate Oligonucleotide
following ConA treatment

| | SEQ ID | ASO Gene | | % mRNA | % mRNA |
|---|---|---|---|---|---|
| ISIS # | NO: | Target | Dose | Expression | Inhibition |
| control | — | — | — | 100% | — |
| 22023 | 73 | coding | 50 mg/kg | 16% | 84% |
| ″ | ″ | ″ | 100 | 18% | 82% |
| | | | mg/kg | | |

### Example 9

Effect of Fas Antisense Oligonucleotides in a Fas Cross-linking Antibody Murine Model for Hepatitis

Injection of agonistic Fas-specific antibody into mice can induce massive hepatocyte apoptosis and liver hemorrhage, and death from acute hepatic failure (Ogasawara, J., et al., Nature, 1993, 364, 806-809). Apoptosis-mediated aberrant cell death has been shown to play an important role in a number of human diseases. For example, in hepatitis, Fas and Fas ligand up-regulated expression are correlated with liver damage and apoptosis. It is thought that apoptosis in the livers of patients with fulminant hepatitis, acute and chronic viral hepatitis, autoimmune hepatitis, as well as chemical or drug induced liver intoxication may result from Fas activation on hepatocytes.

Eight to ten week-old female Balb/c mice were intra peritoneally injected with oligonucleotides 22023 (SEQ ID NO. 73) and 22028 (SEQ ID NO. 78) at 50 mg/kg, daily for 4 days. Four hours after the last dose, 7.5 ?g of mouse Fas antibody (Pharmingen, San Diego, Calif.) was injected into the mice. Mortality of the mice was measured for more than 10 days following antibody treatment.

Results are shown in Table 15. Mortality is expressed as a fraction where the denominator is the total number of mice used and the numerator is the number that died.

TABLE 15
Protective Effects of Fas Antisense Chimeric (deoxy gapped)
Phosphorothioate Oligonucleotides in Fas Antibody Cross-
linking Induced Death in Balb/c Mice

| | SEQ ID | ASO Gene | | Mortalit |
|---|---|---|---|---|
| ISIS # | NO: | Target | Dose | y |
| saline | — | — | — | 6/6 |
| 22023 | 73 | coding | 50 mg/kg | 0/6 |
| 22028 | 78 | coding | 50 mg/kg | 0/6 |

Oligonucleotides 22023 (SEQ ID NO. 73) and 22028 (SEQ ID NO. 78) completely protected the Fas antibody treated mice from death. Mice injected with saline or scrambled control oligonucleotide did not confer any protective effect.

Total RNA was extracted from the livers of Fas antibody treated mice using the RNEASY⁷ kit (Qiagen, Santa Clarita, Calif.). Fas mRNA expression was quantitated using RPA as described in Example 5. It was found that high levels of Fas mRNA expression in this model correlated with increased mortality of Fas antibody treated mice.

### Example 10

Oligonucleotide Synthesis—96 Well Plate Format

In accordance with the present invention additional oligonucleotides targeting human Fas were designed and screened in a 96 well plate format.

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g., PE-Applied Biosystems, Foster City, Calif., or Pharmacia, Piscataway, N.J.). Non-standard nucleosides are synthesized as per known literature or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 11

Oligonucleotide Analysis -96 Well Plate Format

The concentration of oligonucleotide in each well was assessed by dilution of samples and UV absorption spectroscopy. The full-length integrity of the individual products was evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 12

Cell Culture and Oligonucleotide Treatment-96 Well Plate Format

The effect of antisense compounds on target nucleic acid expression can be tested in any of a variety of cell types provided that the target nucleic acid is present at measurable levels. This can be routinely determined using, for example, PCR or Northern blot analysis. The following 5 cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's SA basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence.

NHDF Cells:

Human neonatal dermal fibroblast (NHDF) were obtained from the Clonetics Corporation (Walkersville Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

HepG2 Cells:

The human hepatoblastoma cell line HepG2 was obtained from the American Type Culure Collection (Manassas, Va.). HepG2 cells were routinely cultured in Eagle's MEM supplemented with 10% fetal calf serum, non-essential amino acids, and 1 mM sodium pyruvate (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

Treatment with Antisense Compounds:

When cells reached 80% confluency, they were treated with oligonucleotide. For cells grown in 96-well plates, wells were washed once with 200 μL OPTI-MEM™-1 reduced-serum medium (Gibco BRL) and then treated with 130 μL of OPTI-MEM™-L containing 3.75 μg/mL LIPOFECTINTM (Gibco BRL) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920, TCCGTCATCGCTCCTCAGGG, SEQ ID NO: 86, a 2′-O-methoxyethyl gapmer (2′-O-methoxyethyls shown in bold) with a phosphorothioate backbone which is targeted to human H-ras. For mouse or rat cells the positive control oligonucleotide is ISIS 15770, ATGCATTCTGCCCCCAAGGA, SEQ ID NO: 87, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-Ha-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf MRNA is then utilized as the oligonucleotide screening concentration in subsequent experiments for that cell line. If 60% inhibition is not achieved, that particular cell line is deemed as unsuitable for oligonucleotide transfection experiments.

### Example 13

Analysis of Oligonucleotide Inhibition of Fas expression-96 Well Plate Format

Antisense modulation of Fas expression can be assayed in a variety of ways known in the art. For example, Fas mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels of Fas can be quantitated in a variety of ways well known in the art, such as immunoprecipitation, Western blot analysis (immunoblotting), ELISA or fluorescence-activated cell sorting (FACS). Antibodies directed to Fas can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 14

Poly(A)+ mRNA isolation—96 Well Plate Format

Poly(A)+ mRNA was isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C. was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 15

Total RNA Isolation-96 Well Plate Format

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 100 μL Buffer RLT was added to each well and the plate vigorously agitated for 20 seconds. 100 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 15 seconds. 1 mL of Buffer RWl was added to each well of the RNEASY 96™ plate and the vacuum again applied for 15 seconds. 1 mL of Buffer RPE was then added to each well of the RNEASY 96™ plate and the vacuum applied for a period of 15 seconds. The Buffer RPE wash was then repeated and the vacuum was applied for an additional 10 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 60 μL water into each well, incubating 1 minute, and then applying the vacuum for 30 seconds. The elution step was repeated with an additional 60 μL water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 16

Real-time Quantitative PCR Analysis of Fas mRNA Levels-96 Well Plate Format

Quantitation of Fas MRNA levels was determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif.,or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISMT™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from PE-Applied Biosystems, Foster City, Calif. RT-PCR reactions were carried out by adding 25 μL PCR cocktail (1× TAQMAN™ buffer A, 5.5 mM MgCl₂, 300 μM each of DATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL total RNA solution. The RT reaction was carried out by incubation for 30 minutes at 48° C. Following a 10 minute incubation at 950C to activate the AMPLITAQ GOLD™, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ are taught in Jones, L. J., et al., Analytical Biochemistry, 1998, 265, 368-374.

In this assay, 175 μL of RiboGreen™ working reagent (RiboGreen™ reagent diluted 1:2865 in 10 mM Tris-HCl, 1 mM EDTA, pH 7.5) is pipetted into a 96-well plate containing 25 uL purified, cellular RNA. The plate is read in a CytoFluor 4000 (PE Applied Biosystems) with excitation at 480nm and emission at 520 nm.

Probes and primers to human Fas were designed to hybridize to a human Fas sequence, using published sequence information (GenBank accession number X63717, incorporated herein as SEQ ID NO: 1). For human Fas the PCR primers were: forward primer: TCATGACACTAAGTCAAGTTAAAGGCTTT (SEQ ID NO: 88) reverse primer: TCTTGGACATTGTCATTCTTGATCTC (SEQ ID NO: 89) and the PCR probe was: FAM-ATTTTGGCTTCATTGACACCATTCTTTCGAA-TAMRA (SEQ ID NO: 90) where FAM (PE-Applied Biosystems, Foster City, Calif.) is the fluorescent reporter dye) and TAMRA (PE-Applied Biosystems, Foster City, Calif.) is the quencher dye. For human GAPDH the PCR primers were: forward primer: CAACGGATTTGGTCGTATTGG (SEQ ID NO: 91) reverse primer: GGCAACAATATCCACTTTACCAGAGT (SEQ ID NO: 92) and the PCR probe was: 5′ JOE-CGCCTGGTCACCAGGGCTGCT-TAMPA 3′ (SEQ ID NO: 93) where JOE (PE-Applied Biosystems, Foster City, Calif.) is the fluorescent reporter dye) and TAMRA (PE-Applied Biosystems, Foster City, Calif.) is the quencher dye.

### Example 17

Northern Blot Analysis of Fas mRNA levels-96 Well Plate Format

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then robed using QUICKHYB™ hybridization solution (Stratagene, La Jolla, Calif.) using manufacturer's recommendations for stringent conditions.

To detect human Fas, a human Fas specific probe was prepared by PCR using the forward primer TCATGACACTAAGTCAAGTTAAAGGCTTT (SEQ ID NO: 88) and the reverse primer TCTTGGACATTGTCATTCTTGATCTC (SEQ ID NO: 89). To normalize for variations in loading and transfer efficiency membranes were stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 18

Antisense Inhibition of Human Fas Expression by Chimeric Phosphorothioate Oligonucleotides having 2′-MOE Wings and a Deoxy Gap-96 Well Plate Format

In accordance with the present invention, a series of oligonucleotides were designed to target different regions of the human Fas RNA, using published sequences (GenBank accession number X63717, incorporated herein as SEQ ID NO: 1, GenBank accession number D31968, incorporated herein as SEQ ID NO: 94, GenBank accession number X81336, incorporated herein as SEQ ID NO: 95, GenBank accession number X81337, incorporated herein as SEQ ID NO: 96, GenBank accession number X81338, incorporated herein as SEQ ID NO: 97, GenBank accession number X81339, incorporated herein as SEQ ID NO: 98, GenBank accession number X81340, incorporated herein as SEQ ID NO: 99, GenBank accession number X81341, incorporated herein as SEQ ID NO: 100, GenBank accession number X81342, incorporated herein as SEQ ID NO: 101, and GenBank accession number Z70520, incorporated herein as SEQ ID NO: 102). The oligonucleotides are shown in Table 16. “Target site” indicates the first (5′-most) nucleotide number on the particular target sequence to which the oligonucleotide binds. All compounds in Table 16 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human Fas mRNA levels by quantitative real-time PCR as described in other examples herein. ISIS 119513 and ISIS 17020 have the same nucleotide base sequence and differ only in that the cytidine residues are 5-methylcytidines in ISIS 119513. These two oligonucleotides are therefore both labeled SEQ. ID. NO: 11. Data are averages from two experiments. If present, “N.D.” indicates “no data”.

TABLE 16
Inhibition of human Fas mRNA levels by chimeric
phosphorothioate oligonucleotides having 2′-MOE wings and a
deoxy gap

| | | TARGET | TARGET | | | SEQ ID |
|---|---|---|---|---|---|---|
| ISIS # | REGION | SEQ ID NO | SITE | SEQUENCE | % INHIB | NO |
| 119485 | 5′ UTR | 94 | 1398 | tgaggaaggagtcagggttc | 0 | 103 |
| 119486 | 5′ UTR | 94 | 1510 | ggtggtcaggaggatgggaa | 0 | 104 |
| 119487 | Intron | 94 | 1949 | agccagtctccaacgcctcc | 30 | 105 |
| 119488 | Intron | 94 | 2058 | tgccccgcctgcccagcggg | 31 | 106 |
| 119489 | 5′ UTR | 1 | 2 | acacctgtgtgtcactcttg | 36 | 107 |
| 119490 | 5′ UTR | 1 | 51 | gccaagtcactcgtaaaccg | 27 | 108 |
| 119491 | 5′ UTR | 1 | 190 | aatcctccgaagtgaaagag | 43 | 109 |
| 119492 | Start | 1 | 212 | atgcccagcatggttgttga | 38 | 110 |
| | Codon | | | | | |
| 119493 | Coding | 1 | 241 | acgtaagaaccagaggtagg | 51 | 111 |
| 119494 | Coding | 1 | 265 | ttttggacgataatctagca | 63 | 112 |
| 119495 | Coding | 1 | 407 | ttcctttcacctggaggaca | 53 | 113 |
| 119496 | Coding | 1 | 419 | cagtccctagctttcctttc | 36 | 114 |
| 119497 | Coding | 1 | 538 | agccatgtccttcatcacaC | 71 | 115 |
| 119498 | Coding | 1 | 635 | gggtcacagtgttcacatac | 52 | 116 |
| 119499 | Coding | 1 | 687 | gttgctggtgagtgtgcatt | 37 | 117 |
| 119500 | Coding | 1 | 785 | acttcctttctcttcaccca | 0 | 118 |
| 119501 | Coding | 1 | 821 | tggttttcctttctgtgctt | 60 | 119 |
| 119502 | Coding | 1 | 848 | tttaaggttggagattcatg | 22 | 120 |
| 119503 | Coding | 1 | 850 | gatttaaggttggagattca | 3 | 121 |
| 119504 | Coding | 1 | 862 | ccactgtttcaggatttaag | 14 | 122 |
| 119505 | Coding | 1 | 885 | gtcaacatcagataaattta | 26 | 123 |
| 119506 | Coding | 1 | 894 | tttactcaagtcaacatcag | 52 | 124 |
| 119507 | Coding | 1 | 928 | ttagtgtcatgactccagca | 67 | 125 |
| 119508 | Coding | 1 | 936 | aacttgacttagtgtcatga | 31 | 126 |
| 119509 | Coding | 1 | 1039 | tacgaagcagttgaactttc | 51 | 127 |
| 119510 | Coding | 1 | 1097 | ttgagatctttaatcaatgt | 60 | 128 |
| 119511 | Coding | 1 | 1152 | gtccttgaggatgatagtct | 44 | 129 |
| 119512 | Coding | 1 | 1197 | ttggatttcatttctgaagt | 37 | 130 |
| 119513 | Stop | 1 | 1214 | tcactctagaccaagctttg | 66 | 11 |
| | Codon | | | | | |
| 119514 | Stop | 1 | 1218 | tttttcactctagaccaagc | 37 | 131 |
| | Codon | | | | | |
| 119515 | 3′ UTR | 1 | 1291 | aagcagtatttacagccagc | 80 | 132 |
| 119516 | 3′ UTR | 1 | 1331 | tcagcgctaataaatgataa | 54 | 133 |
| 119517 | 3′ UTR | 1 | 1335 | ctcttcagcgctaataaatg | 72 | 134 |
| 119518 | 3′ UTR | 1 | 1437 | atgccactgcatttactctt | 34 | 135 |
| 119519 | 3′ UTR | 1 | 1438 | catgccactgcatttactct | 49 | 136 |
| 119520 | 3′ UTR | 1 | 1525 | acattcatactacagaatca | 52 | 137 |
| 119521 | 3′ UTR | 1 | 1537 | catacactgattacattcat | 17 | 138 |
| 119522 | 3′ UTR | 1 | 1726 | ttacataaatatgatcttct | 32 | 139 |
| 119523 | 3′ UTR | 1 | 1769 | gaggtagagccttatttaaa | 69 | 140 |
| 119524 | 3′ UTR | 1 | 1806 | gtataatatgacaccaataa | 75 | 141 |
| 119525 | 3′ UTR | 1 | 1812 | aatattgtataatatgacac | 15 | 142 |
| 119526 | 3′ UTR | 1 | 1828 | gtgaattcacaattgaaata | 39 | 143 |
| 119527 | 3′ UTR | 1 | 1850 | attataatttaatgttttct | 0 | 144 |
| 119528 | 3′ UTR | 1 | 1940 | tactctcctgctcaaaatgc | 18 | 145 |
| 119529 | 3′ UTR | 1 | 2047 | tggtggactattaagtattt | 67 | 146 |
| 119530 | 3′ UTR | 1 | 2102 | agagcagttagtatctccaa | 78 | 147 |
| 119531 | 3′ UTR | 1 | 2119 | caaagctactttctctgaga | 62 | 148 |
| 119532 | 3′ UTR | 1 | 2128 | gacatgtcacaaagctactt | 41 | 149 |
| 119533 | 3′ UTR | 1 | 2159 | ttatcatctttgattgcaaa | 63 | 150 |
| 119534 | 3′ UTR | 1 | 2210 | atgggacattattgaacatt | 57 | 151 |
| 119535 | 3′ UTR | 1 | 2371 | attcacatttaatacaaact | 0 | 152 |
| 119536 | 3′ UTR | 1 | 2392 | atataaatattatttcttaa | 14 | 153 |
| 119537 | 3′ UTR | 95 | 361 | ctatgtgctactcctaactg | 66 | 154 |
| 119538 | 3′ UTR | 95 | 367 | tgattactatgtgctactcc | 45 | 155 |
| 119539 | 3′ UTR | 95 | 469 | tataaataaaactcatcttt | 0 | 156 |
| 119540 | 3′ UTR | 96 | 384 | cttccctttcctgtgtgtca | 50 | 157 |
| 119541 | 3′ UTR | 96 | 401 | taccctagccacctgtcctt | 12 | 158 |
| 119542 | 3′ UTR | 96 | 470 | ctggaagaattgcctagact | 39 | 159 |
| 119543 | 3′ UTR | 96 | 492 | atatttactcattctcctat | 10 | 160 |
| 119544 | 3′ UTR | 96 | 808 | atgtccagaggtttcttcat | 54 | 161 |
| 119545 | 3′ UTR | 96 | 851 | agaaacattgctttataggc | 61 | 162 |
| 119546 | 5′ UTR | 97 | 7 | atgacaccagtaatacagtc | 58 | 163 |
| 119547 | 5′ UTR | 97 | 41 | tttgagatccactgcttata | 7 | 164 |
| 119548 | 5′ UTR | 97 | 114 | gtttggaaactattagttat | 15 | 165 |
| 119549 | Intron | 98 | 33 | atgtgtgatttcottcagac | 49 | 166 |
| 119550 | Intron | 98 | 338 | atcataaggaatgactgtct | 46 | 167 |
| 119551 | Intron | 98 | 470 | aatggcactttgtaaattag | 50 | 168 |
| 119552 | Intron | 98 | 480 | tataattttcaatggcactt | 15 | 169 |
| 119553 | Intron | 98 | 494 | cagaataattcctttataat | 16 | 170 |
| 119554 | Coding | 98 | 543 | ccatgttcacatctagaaaa | 30 | 171 |
| 119555 | Start | 99 | 67 | tctcttcactgaaagaacaa | 17 | 172 |
| | Codon | | | | | |
| 119556 | 3′ UTR | 99 | 172 | aggaaagctgatacctattt | 47 | 173 |
| 119557 | 3′ UTR | 100 | 293 | catctctatgaaataaaatg | 3 | 174 |
| 119558 | 3′ UTR | 100 | 504 | ggaaaagtttcttaagcctc | 60 | 175 |
| 119559 | 3′ UTR | 100 | 656 | ttatctctaaatcacagatc | 57 | 176 |
| 119560 | 3′ UTR | 101 | 1759 | aaagagaaaaccagaaatac | 0 | 177 |
| 119561 | 3′ UTR | 110 | 1804 | gttagagaaaaggaagacaa | 56 | 178 |
| 119562 | Coding | 102 | 325 | atgttcacatcatgtccttc | 5 | 179 |

As shown in Table 16, SEQ ID NOs 11, 105, 106, 107, 108, 109, 110, 111, 112, 113, 114, 115, 116, 117, 119, 123, 124, 125, 126, 127, 128, 129, 130, 131, 132, 133, 134, 135, 136, 137, 139, 140, 141, 143, 146, 147, 148, 149, 150, 151, 154, 155, 157, 159, 161, 162, 163, 166, 167, 168, 171, 173, 175, 176 and 178 demonstrated at least 25% inhibition of human Fas expression in this assay and are therefore preferred. The target sites to which these preferred sequences are complementary are herein referred to as “active sites” and are therefore preferred sites for targeting by compounds of the present invention.

### Example 19

Western Blot Analysis of Fas Protein Levels

Western blot analysis (immunoblot analysis) is carried out using standard methods. Cells are harvested 16-20 hours after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to Fas is used, with a radiolabelled or fluorescently labeled secondary antibody directed against the primary antibody species. Bands are visualized using a PHOSPHORIMAGER™ (Molecular Dynamics, Sunnyvale Calif.).

### Example 20

Effect of Fas Antisense Oligonucleotides in a Murine Model of Renal Ischemia-Reperfusion Injury

Ischemia-reperfusion can result in organ failure with the severity of damage being proportional to the duration of ischemia. In the kidney, the damage has been partially attributed to apoptosis in tubular cells. It has been demonstrated that Fas mRNA expression is upregulated in kidneys suffering from ischemia-reperfusion injury. Mice lacking Fas (lpr mice) undergo significantly less apoptosis and it is therefore believed that the apoptotic response to ischemia-reperfusion involves the Fas/FasL signaling pathway. Consequently, inhibition of Fas might serve to attenuate organ injury in the kidney after ischemia-reperfusion.

The antisense oligonucleotide ISIS 22023 (SEQ ID NO: 73) and an 8-base mismatch control oligonucleotide (ISIS 29837; SEQ ID NO: 180) were used in studies to determine the effects of systemic administration of anti-Fas antisense oligonucleotides on renal ischemia-reperfusion injury.

Male C57BL/10 (B10) mice (10 to 12 weeks old purchased from The Jackson Laboratory) were injected intraperitoneally with ISIS 22023 or the control oligonucleotide at 25-100 mg/day for five days. Following the treatment protocol, the left renal artery and vein were clamped for 30, 60 or 90 minutes followed by reperfusion for 24 hours. Serum was collected and both kidneys were removed. The right kidneys were used as controls. Reperfusion damage was determined by histological examination. Apoptosis and DNA fragmentation in the renal cells was identified by in situ TdT-mediated dUTP nick end labeling (TUNEL) staining and agarose-gel electrophoresis. Methods of detecting DNA fragmentation by TUNEL analysis is well known in the art. Briefly, the cleavage of cellular DNA into low molecular weight fragments, which occurs in the early stages of apoptosis, is detected by labeling the free 3′-OH termini of the fragments with modified nucleotides. These nucleotides usually contain a chromophore or fluorescent detectible group and are added by the enzyme, terminal deoxynucleotidyl transferase (TdT) to the blunt ends of DNA fragments. Cells are then analyzed for the presence of the chromophore using standard electrophoresis and imaging techniques. Cells undergoing apoptosis are considered TUNEL-positive.

Expression of Fas was determined by immunohistochemical staining and Western blotting using antibodies specific for Fas. Serum BUN and creatinine were also measured in a sub-group of mice that underwent clamping of both renal arteries.

Histological examination revealed that histological damage caused by ischemia-reperfusion was significantly reduced by administration of the Fas antisense oligonucleotide at doses higher than 50 mg/day including attenuated tubule cell detachment and cast formation.

Immunohistochemistry revealed that normal kidneys expressed Fas, particularly in the tubule areas, but few cells were TUNEL positive. This is believed to indicate that the renal tubule cells are vulnerable to Fas-mediated apoptosis.

Ischemia longer than 60 minutes significantly enhanced Fas expression and apoptotic activity in tubular cells, both of which were significantly inhibited by systemic administration of ISIS 22023.

TUNEL positive cells reached up to 51.7+/−4.9 hpf in kidneys that were ischemic for 90 minutes (compared with 0 hpf in normal kidneys), while only 9.3+/−1.7 positive cells were identified in animals that were treated with ISIS 22023 at 50 mg/day.

Taken together these data suggest that inhibition of Fas expression and apoptotic activity in ischemic kidneys by systemic treatment with antisense oligonucleotide administration is a potential therapeutic approach as well as being a convenient mode of delivery.

What is claimed is:

1. An antisense compound 8 to 30 nucleobases in length targeted to the 5′-untranslated region, translational start site, translational termination region or 3′-untranslated region of a nucleic acid molecule encoding Fas, wherein said antisense compound inhibits the expression of said Fas.

2. The antisense compound of claim 1 which is an antisense oligonucleotide.

3. The antisense compound of claim 2 wherein the antisense oligonucleotide has a sequence comprising SEQ ID NO: 5, 11, 12, 13, 14, 15, 16, 17, 19, 20, 21, 67, 68, 80, 82, 105, 106, 107, 108, 109, 110, 131, 132, 133, 134, 135, 136, 137, 139, 140, 141, 143, 146, 147, 148, 149, 150, 151, 154, 155, 157, 159, 161, 162, 163, 166, 167, 168, 173, 175, 176 or 178.

4. The antisense compound of claim 2 wherein the antisense oligonucleotide comprises at least one modified internucleoside linkage.

5. The antisense compound of claim 4 wherein the modified internucleoside linkage is a phosphorothioate linkage.

6. The antisense compound of claim 2 wherein the antisense oligonucleotide comprises at least one modified sugar moiety.

7. The antisense compound of claim 6 wherein the modified sugar moiety is a 2′-O-methoxyethyl moiety.

8. The antisense compound of claim 2 wherein the antisense oligonucleotide comprises at least one modified nucleobase.

9. The antisense compound of claim 8 wherein modified nucleobase is a 5-methyl cytosine.

10. The antisense compound of claim 2 wherein the antisense oligonucleotide is a chimeric oligonucleotide.

11. A pharmaceutical composition comprising the antisense compound of claim 1 and a pharmaceutically acceptable carrier or diluent.

12. The pharmaceutical composition of claim 11 further comprising a colloidal dispersion system.

13. The pharmaceutical composition of claim 11 wherein the antisense compound is an antisense oligonucleotide.

14. A method of inhibiting the expression of Fas in cells or tissues comprising contacting said cells or tissue with the antisense compound of claim 1 so that expression of Fas is inhibited.

15. A method of treating an animal having a disease or condition associated with Fas comprising administering to said animal a therapeutically or prophylactically effective amount of the antisense compound of claim 1 so that expression of Fas is inhibited.

16. The method of claim 15 wherein the disease or condition is an autoimmune or inflammatory disease.

17. The method of claim 16 wherein said inflammatory or autoimmune disease or condition is hepatitis.

18. The method of claim 15 wherein said disease or condition is cancer.

19. The method of claim 18 wherein said cancer is a cancer of the colon, liver, lung or a lymphoma.

20. The method of claim 15 wherein the disease or condition is associated with apoptosis.

21. The method of claim 15 wherein the disease or condition is allograft rejection.

22. The method of claim 15 wherein the disease or condition is ischemia reperfusion injury.

23. An antisense compound 8 to 30 nucleobases in length targeted to the coding region of a nucleic acid molecule encoding Fas, wherein said antisense compound inhibits the expression of said Fas and has a sequence comprising SEQ ID NO: 6, 7, 8, 10, 69, 73, 74, 76, 78, 111, 112, 113, 114, 115, 116, 117, 119, 123, 124, 125, 126, 127, 128, 129, 130 or 171.

24. The antisense compound of claim 23 which is an antisense oligonucleotide.

25. The antisense compound of claim 24 wherein the antisense oligonucleotide comprises at least one modified internucleoside linkage.

26. The antisense compound of claim 25 wherein the modified internucleoside linkage is a phosphorothioate linkage.

27. The antisense compound of claim 24 wherein the antisense oligonucleotide comprises at least one modified sugar moiety.

28. The antisense compound of claim 27 wherein the modified sugar moiety is a 2′-O-methoxyethyl moiety.

29. The antisense compound of claim 24 wherein the antisense oligonucleotide comprises at least one modified nucleobase.

30. The antisense compound of claim 29 wherein modified nucleobase is a 5-methyl cytosine.

31. The antisense compound of claim 24 wherein the antisense oligonucleotide is a chimeric oligonucleotide.

32. A pharmaceutical composition comprising the antisense compound of claim 23 and a pharmaceutically acceptable carrier or diluent.

33. The pharmaceutical composition of claim 32 further comprising a colloidal dispersion system.

34. The pharmaceutical composition of claim 32 wherein the antisense compound is an antisense oligonucleotide.

35. A method of inhibiting the expression of Fas in cells or tissues comprising contacting said cells or tissue with the antisense compound of claim 23 so that expression of Fas is inhibited.

36. A method of treating an animal having a disease or condition associated with Fas comprising administering to said animal a therapeutically or prophylactically effective amount of the antisense compound of claim 23 so that expression of Fas is inhibited.

37. The method of claim 36 wherein the disease or condition is an autoimmune or inflammatory disease.

38. The method of claim 37 wherein said inflammatory or autoimmune disease or condition is hepatitis.

39. The method of claim 36 wherein said disease or condition is cancer.

40. The method of claim 39 wherein said cancer is a cancer of the colon, liver, lung or a lymphoma.

41. The method of claim 36 wherein the disease or condition is associated with apoptosis.

42. The method of claim 36 wherein the disease or condition is allograft rejection.

43. The method of claim 36 wherein the disease or condition is ischemia reperfusion injury.

44. An antisense compound 8 to 30 nucleobases in length targeted to the 5′-untranslated region, translational termination region, or 3′ untranslated region of a nucleic acid molecule encoding Fas ligand, wherein said antisense compound inhibits the expression of said Fas ligand.

45. The antisense compound of claim 44 which is an antisense oligonucleotide.

46. The antisense compound of claim 45 wherein the antisense oligonucleotide has a sequence comprising SEQ ID NO: 36, 37, 43 or 44.

47. The antisense compound of claim 45 wherein the antisense oligonucleotide comprises at least one modified internucleoside linkage.

48. The antisense compound of claim 47 wherein the modified internucleoside linkage is a phosphorothioate linkage.

49. The antisense compound of claim 45 wherein the antisense oligonucleotide comprises at least one modified sugar moiety.

50. The antisense compound of claim 49 wherein the modified sugar moiety is a 2′-O-methoxyethyl moiety.

51. The antisense compound of claim 45 wherein the antisense oligonucleotide comprises at least one modified nucleobase.

52. The antisense compound of claim 51 wherein modified nucleobase is a 5-methyl cytosine.

53. The antisense compound of claim 45 wherein the antisense oligonucleotide is a chimeric oligonucleotide.

54. A pharmaceutical composition comprising the antisense compound of claim 44 and a pharmaceutically acceptable carrier or diluent.

55. The pharmaceutical composition of claim 54 further comprising a colloidal dispersion system.

56. The pharmaceutical composition of claim 54 wherein the antisense compound is an antisense oligonucleotide.

57. A method of inhibiting the expression of Fas ligand in cells or tissues comprising contacting said cells or tissue with the antisense compound of claim 44 so that expression of Fas ligand is inhibited.

58. A method of treating an animal having a disease or condition associated with Fas ligand comprising administering to said animal a therapeutically or prophylactically effective amount of the antisense compound of claim 44 so that expression of Fas ligand is inhibited.

59. The method of claim 58 wherein the disease or condition is an autoimmune or inflammatory disease.

60. The method of claim 59 wherein said inflammatory or autoimmune disease or condition is hepatitis.

61. The method of claim 58 wherein said disease or condition is cancer.

62. The method of claim 61 wherein said cancer is a cancer of the colon, liver, lung or a lymphoma.

63. A method of preventing allograft rejection in an allograft recipient comprising administering to the allograft recipient an antisense compound 8 to 50 nucleobases in length targeted to a nucleic acid sequence encoding Fas.

64. The method of claim 63 wherein the antisense compound is an antisense oligonucleotide.

65. The method of claim 64 wherein the antisense oligonucleotide comprises SEQ ID NO: 73.

66. The method of claim 63 wherein the allograft is a cardiac allograft.

67. The method of claim 63 wherein the allograft is a renal allograft.

68. The method of claim 63 wherein the allograft is an hepatic allograft.

69. The method of claim 63 wherein the allograft is a skin allograft.

70. A method of preventing rejection of an allograft by an allograft recipient comprising contacting the allograft with an antisense compound 8 to 50 nucleobases in length targeted to a nucleic acid sequence encoding Fas.

71. The method of claim 70 wherein the perfusion is performed ex vivo.

72. The method of claim 70 wherein the antisense compound is an antisense oligonucleotide.

73. The method of claim 70 wherein the antisense oligonucleotide comprises SEQ ID NO: 73.

74. The method of claim 70 wherein the allograft is a cardiac allograft.

75. The method of claim 70 wherein the allograft is a renal allograft.

76. The method of claim 70 wherein the allograft is an hepatic allograft.

77. The method of claim 70 wherein the allograft is a skin allograft.

78. A method of preventing ischemia reperfusion injury in an allograft recipient comprising administering to the allograft recipient an antisense compound 8 to 50 nucleobases in length targeted to a nucleic acid sequence encoding Fas.

79. The method of claim 78 wherein the antisense compound is an antisense oligonucleotide.

80. The method of claim 79 wherein the antisense oligonucleotide comprises SEQ ID NO: 73.

81. The method of claim 78 wherein the allograft is a cardiac allograft.

82. The method of claim 78 wherein the allograft is a renal allograft.

83. The method of claim 78 wherein the allograft is an hepatic allograft.

84. The method of claim 78 wherein the allograft is a skin allograft.

85. A method of preventing ischemia reperfusion injury of an allograft comprising contacting the allograft with an antisense compound 8 to 50 nucleobases in length targeted to a nucleic acid sequence encoding Fas.

86. The method of claim 85 wherein the perfusion is performed ex vivo.

87. The method of claim 85 wherein the antisense compound is an antisense oligonucleotide.

88. The method of claim 87 wherein the antisense oligonucleotide comprises SEQ ID NO: 73.

89. The method of claim 85 wherein the allograft is a cardiac allograft.

90. The method of claim 85 wherein the allograft is a renal allograft.

91. The method of claim 85 wherein the allograft is an hepatic allograft.

92. The method of claim 85 wherein the allograft is a skin allograft.

93. A method of preventing apoptosis in an allograft recipient comprising administering to the allograft recipient an antisense compound 8 to 50 nucleobases in length targeted to a nucleic acid sequence encoding Fas.

94. The method of claim 93 wherein the antisense compound is an antisense oligonucleotide.

95. The method of claim 94 wherein the antisense oligonucleotide comprises SEQ ID NO: 73.

96. The method of claim 93 wherein the allograft is a cardiac allograft.

97. The method of claim 93 wherein the allograft is a renal allograft.

98. The method of claim 93 wherein the allograft is an hepatic allograft.

99. The method of claim 93 wherein the allograft is a skin allograft.

100. A method of preventing apoptosis in an allograft comprising contacting the allograft with an antisense compound 8 to 50 nucleobases in length targeted to a nucleic acid sequence encoding Fas.

101. The method of claim 100 wherein the perfusion is performed ex vivo.

102. The method of claim 100 wherein the antisense compound is an antisense oligonucleotide.

103. The method of claim 102 wherein the antisense oligonucleotide comprises SEQ ID NO: 73.

104. The method of claim 100 wherein the allograft is a cardiac allograft.

105. The method of claim 100 wherein the allograft is a renal allograft.

106. The method of claim 100 wherein the allograft is an hepatic allograft.

107. The method of claim 100 wherein the allograft is a skin allograft.

108. An antisense compound 8 to 30 nucleobases in length targeted to a nucleic acid molecule encoding Fap-1, wherein said antisense compound inhibits the expression of said Fap-1.

109. The antisense compound of claim 108 which is an antisense oligonucleotide.

110. The antisense compound of claim 109 wherein the antisense oligonucleotide has a sequence comprising SEQ ID NO: 48, 50, 51, 52, 53, 58, 59, 60, or 64.

111. The antisense compound of claim 109 wherein the antisense oligonucleotide compri ses at least one modif ied internucleoside linkage.

112. The antisense compound of claim 111 wherein the modified internucleoside linkage is a phosphorothioate linekage.

113. The antisense compound of claim 109 wherein the antisense oligonucleotide comprises at least one modified sugar moiety.

114. The antisense compound of claim 113 wherein the modified sugar moiety is a 2′-O-methoxyethyl moiety.

115. The antisense compound of claim 109 wherein the antisense oligonucleotide comprises at least one modified nucleobase.

116. The antisense compound of claim 115 wherein the modified nucleobase is a 5-methyl cytosine.

117. The antisense compound of claim 115 wherein the antisense oligonucleotide is a chimeric oligonucleotide.

118. A pharmaceutical composition comprising the antisense compound of claim 108 and a pharmaceutically acceptable carrier or diluent.

119. The pharmaceutical composition of claim 118 further comprising a colloidal dispersion system.

120. The pharmaceutical composition of claim 118 wherein the antisense compound is an antisense oligonucleotide.

121. A method of inhibiting the expression of Fap-1 in cells or tissues comprising contacting said cells or tissue with the antisense compound of claim 108 so that expression of Fap-1 is inhibited.

122. A method of treating an animal having a disease or condition associated with Fap-1 comprising administering to said animal a therapeutically or prophylactically effective amount of the antisense compound of claim 108 so that expression of Fap-1 is inhibited.

123. The method of claim 122 wherein the disease or condition is an autoimmune or inflammatory disease.

124. The method of claim 123 wherein said inflammatory or autoimmune disease or condition is hepatitis.

125. The method of claim 122 wherein said disease or condition is cancer.

126. The method of claim 125 wherein said cancer is a cancer of the colon, liver, lung or a lymphoma.


SEQUENCE LISTING (180 sequences)

SEQ ID NO: 1 - DNA, 2551 bp, Homo sapiens
  CDS (221)..(1228)
  ref <303>: JOURNAL: J. Biol. Chem.
  ref <304>: VOLUME: 267
  ref <305>: ISSUE: 15
  ref <306>: PAGES: 10709-10715
  ref <307>: DATE: 1992-05-25
  ref <308>: DATABASE ACCESSION NUMBER: X63717/Genbank
  ref <309>: DATABASE ENTRY DATE: 1996-07-19
gcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaata

SEQ ID NO: 2 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 3 - DNA, 20 bp, Artificial Sequence
  Synthetic Sequence
cgtaaaccgcttccctcact

SEQ ID NO: 4 - DNA, 20 bp, Artificial Sequence
  Synthetic Sequence
gtgttccgtgccagtgcccg

SEQ ID NO: 5 - DNA, 20 bp, Artificial Sequence
  Synthetic Sequence
gcccagcatggttgttgagc

SEQ ID NO: 6 - DNA, 20 bp, Artificial Sequence
  Synthetic Sequence
cttcctcaattccaatccct

SEQ ID NO: 7 - DNA, 20 bp, Artificial Sequence
  Synthetic Sequence
cttcttggcagggcacgcag

SEQ ID NO: 8 - DNA, 20 bp, Artificial Sequence
  Synthetic Sequence
tgcacttggtattctgggtc

SEQ ID NO: 9 - DNA, 20 bp, Artificial Sequence
  Synthetic Sequence
gctggtgagtgtgcattcct

SEQ ID NO: 10 - DNA, 20 bp, Artificial Sequence
  Synthetic Sequence
cattgacaccattctttcga

SEQ ID NO: 11 - DNA, 20 bp, Artificial Sequence
  Synthetic Sequence
tcactctagaccaagctttg

SEQ ID NO: 12 - DNA, 20 bp, Artificial Sequence
  Synthetic Sequence
cccagtaaaaaaccaagcag

SEQ ID NO: 13 - DNA, 20 bp, Artificial Sequence
  Synthetic Sequence
tatgttggctcttcagcgct

SEQ ID NO: 14 - DNA, 20 bp, Artificial Sequence
  Synthetic Sequence
atttgggtacttagcatgcc

SEQ ID NO: 15 - DNA, 20 bp, Artificial Sequence
  Synthetic Sequence
gggttagcctgtggatagac

SEQ ID NO: 16 - DNA, 20 bp, Artificial Sequence
  Synthetic Sequence
caaagtggcctgcctgttca

SEQ ID NO: 17 - DNA, 20 bp, Artificial Sequence
  Synthetic Sequence
ttgagccagtaaaatgcata

SEQ ID NO: 18 - DNA, 20 bp, Artificial Sequence
  Synthetic Sequence
tgagcaccaaggcaaaaatg

SEQ ID NO: 19 - DNA, 20 bp, Artificial Sequence
  Synthetic Sequence
tcttgccttttggtggacta

SEQ ID NO: 20 - DNA, 20 bp, Artificial Sequence
  Synthetic Sequence
agcaggttttacatgggaca

SEQ ID NO: 21 - DNA, 20 bp, Artificial Sequence
  Synthetic Sequence
ggtatgacaagagcaattcc

SEQ ID NO: 22 - DNA, 20 bp, Artificial Sequence
  Synthetic Sequence
ggtggttccaggtatctgct

SEQ ID NO: 23 - DNA, 20 bp, Artificial Sequence
  Synthetic Sequence
tataattccaaacacaaggg

SEQ ID NO: 24 - DNA, 1890 bp, Homo sapiens
  CDS (189)..(1034)
  ref <303>: JOURNAL: Biochim. Biophys. Acta
  ref <304>: VOLUME: 204
  ref <305>: ISSUE: 2
  ref <306>: PAGES: 468-474
  ref <307>: DATE: 1994-10-28
  ref <308>: DATABASE ACCESSION NUMBER: D31822/Genbank
  ref <309>: DATABASE ENTRY DATE: 1999-02-08
aaacagagagagatagagaaagagaaagacagaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctacccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgccaattgtaggggtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaat

SEQ ID NO: 25 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 26 - DNA, 20 bp, Artificial Sequence
  Synthetic Sequence
ccatagctaagggaaacacc

SEQ ID NO: 27 - DNA, 20 bp, Artificial Sequence
  Synthetic Sequence
gccagccccagcaaacggtt

SEQ ID NO: 28 - DNA, 20 bp, Artificial Sequence
  Synthetic Sequence
tgcatggcagctggtgagtc

SEQ ID NO: 29 - DNA, 20 bp, Artificial Sequence
  Synthetic Sequence
ggaagaactgtgcctggagg

SEQ ID NO: 30 - DNA, 20 bp, Artificial Sequence
  Synthetic Sequence
tggcagcggtagtggaggca

SEQ ID NO: 31 - DNA, 20 bp, Artificial Sequence
  Synthetic Sequence
gctgtgtgcatctggctggt

SEQ ID NO: 32 - DNA, 20 bp, Artificial Sequence
  Synthetic Sequence
aatgggccactttcctcagc

SEQ ID NO: 33 - DNA, 20 bp, Artificial Sequence
  Synthetic Sequence
gcaggttgttgcaagattga

SEQ ID NO: 34 - DNA, 20 bp, Artificial Sequence
  Synthetic Sequence
aagattgaacactgccccca

SEQ ID NO: 35 - DNA, 20 bp, Artificial Sequence
  Synthetic Sequence
aatcccaaagtgcttctctt

SEQ ID NO: 36 - DNA, 20 bp, Artificial Sequence
  Synthetic Sequence
ttctcggtgcctgtaacaaa

SEQ ID NO: 37 - DNA, 20 bp, Artificial Sequence
  Synthetic Sequence
gctacagacattttgaaccc

SEQ ID NO: 38 - DNA, 20 bp, Artificial Sequence
  Synthetic Sequence
ccgtcatattcctccatttg

SEQ ID NO: 39 - DNA, 20 bp, Artificial Sequence
  Synthetic Sequence
ccctcttcacatggcagccc

SEQ ID NO: 40 - DNA, 20 bp, Artificial Sequence
  Synthetic Sequence
ggtgtccttttcaatctgcc

SEQ ID NO: 41 - DNA, 20 bp, Artificial Sequence
  Synthetic Sequence
cagtcccccttgaggtagca

SEQ ID NO: 42 - DNA, 20 bp, Artificial Sequence
  Synthetic Sequence
gtgaagatgctgccagtggg

SEQ ID NO: 43 - DNA, 20 bp, Artificial Sequence
  Synthetic Sequence
cccctacaattggcactgga

SEQ ID NO: 44 - DNA, 20 bp, Artificial Sequence
  Synthetic Sequence
tcttgaccaaatgcaaccca

SEQ ID NO: 45 - DNA, 8119 bp, Homo sapiens
  CDS (64)..(7521)
  ref <303>: JOURNAL: FEBS Lett.
  ref <304>: VOLUME: 337
  ref <305>: ISSUE: 2
  ref <306>: PAGES: 200-206
  ref <307>: DATE: 1994-01-10
  ref <308>: DATABASE ACCESSION NUMBER: D21209/Genbank
  ref <309>: DATABASE ENTRY DATE: 1999-02-05
cgtccctgcagccctcgcccggcgctccagtagcaggacccggtctcgggaccagccggtaatatgcacgtgtcactagctgaggccctggaggttcggggtggaccacttcaggaggaagaaatatgggctgtattaaatcaaagtgctgaaagtctccaagaattattcagaaaagtaagcctagctgatcctgctgcccttggcttcatcatttctccatggtctctgctgttgctgccatctggtagtgtgtcatttacagatgaaaatatttccaatcaggatcttcgagcattcactgcaccagaggttcttcaaaatcagtcactaacttctctctcagatgttgaaaagatccacatttattctcttggaatgacactgtattggggggctgattatgaagtgcctcagagccaacctattaagcttggagatcatctcaacagcatactgcttggaatgtgtgaggatgttatttacgctcgagtttctgttcggactgtgctggatgcttgcagtgcccacattaggaatagcaattgtgcaccctcattttcctacgtgaaacacttggtaaaactggttctgggaaatctttctgggacagatcagctttcctgtaacagtgaacaaaagcctgatcgaagccaggctattcgagatcgattgcgaggaaaaggattaccaacaggaagaagctctacttctgatgtactagacatacaaaagcctccactctctcatcagacctttcttaacaaagggcttagtaaatctatgggatttctgtccatcaaagatacacaagatgagaattatttcaaggacattttatcagataattctggacgtgaagattctgaaaatacattctccccttaccagttcaaaactagtggcccagaaaaaaaacccatccctggcattgatgtgctttctaagaagaagatctgggcttcatccatggacttgctttgtacagctgacagagacttctcttcaggagagactgccacatatcgtcgttgtcaccctgaggcagtaacagtgcggacttcaactactcctagaaaaaaggaggcaagatactcagatggaagtatagccttggatatctttggccctcagaaaatggatccaatatatcacactcgagaattgcccacctcctcagcaatatcaagtgctttggaccgaatccgagagagacaaaagaaacttcaggttctgagggaagccatgaatgtagaagaaccagttcgaagatacaaaacttatcatggtgatgtctttagtacctccagtgaaagtccatctattatttcctctgaatcagatttcagacaagtgagaagaagtgaagcctcaaagaggtttgaatccagcagtggtctcccaggggtagatgaaaccttaagtcaaggccagtcacagagaccgagcagacaatatgaaacaccctttgaaggcaacttaattaatcaagagatcatgctaaaacggcaagaggaagaactgatgcagctacaagccaaaatggcccttagacagtctcggttgagcctatatccaggagacacaatcaaagcgtccatgcttgacatcaccagggatccgttaagagaaattgccctagaaacagccatgactcaaagaaaactgaggaatttctttggccctgagtttgtgaaaatgacaattgaaccatttatatctttggatttgccacggtctattcttactaagaaagggaagaatgaggataaccgaaggaaagtaaacataatgcttctgaacgggcaaagactggaactgacctgtgataccaaaactatatgtaaagatgtgtttgatatggttgtggcacatattggcttagtagagcatcatttgtttgctttagctaccctcaaagataatgaatatttctttgttgatcctgacttaaaattaaccaaagtggccccagagggatggaaagaagaaccaaagaaaaagaccaaagccactgttaattttactttgtttttcagaattaaattttttatggatgatgttagtctaatacaacatactctgacgtgtcatcagtattaccttcagcttcgaaaagatattttggaggaaaggatgcactgtgatgatgagacttccttattgctggcatccttggctctccaggctgagtatggagattatcaaccagaggttcatggtgtgtcttactttagaatggagcactatttgcccgccagagtgatggagaaacttgatttatcctatatcaaagaagagttacccaaattgcataatacctatgtgggagcttctgaaaaagagacagagttagaatttttaaaggtctgccaaagactgacagaatatggagttcattttcaccgagtgcaccctgagaagaagtcacaaacaggaatattgcttggagtctgttctaaaggtgtccttgtgtttgaagttcacaatggagtgcgcacattggtccttcgctttccatggagggaaaccaagaaaatatctttttctaaaaagaaaatcacattgcaaaatacatcagatggaataaaacatggcttccagacagacaacagtaagatatgccagtacctgctgcacctctgctcttaccagcataagttccagctacagatgagagcaagacagagcaaccaagatgcccaagatattgagagagcttcgtttaggagcctgaatctccaagcagagtctgttagaggatttaatatgggacgagcaatcagcactggcagtctggccagcagcaccctcaacaaacttgctgttcgacctttatcagttcaagctgagattctgaagaggctatcctgctcagagctgtcgctttaccagccattgcaaaacagttcaaaagagaagaatgacaaagcttcatgggaggaaaagcctagagagatgagtaaatcataccatgatctcagtcaggcctctctctatccacatcggaaaaatgtcattgttaacatggaacccccaccacaaaccgttgcagagttggtgggaaaaccttctcaccagatgtcaagatctgatgcagaatctttggcaggagtgacaaaacttaataattcaaagtctgttgcgagtttaaatagaagtcctgaaaggaggaaacatgaatcagactcctcatccattgaagaccctgggcaagcatatgttctaggaatgactatgcatagttctggaaactcttcatcccaagtacccttaaaagaaaatgatgtgctacacaaaagatggagcatagtatcttcaccagaaagggagatcaccttagtgaacctgaaaaaagatgcaaagtatggcttgggatttcaaattattggtggggagaagatgggaagactggacctaggcatatttatcagttcagttgcccctggaggaccagctgacttggatggatgcttgaagccaggagaccgtttgatatctgtgaatagtgtgagtctggagggagtcagccaccatgctgcaattgaaattttgcaaaatgcacctgaagatgtgacacttgttatctctcagccaaaagaaaagatatccaaagtgccttctactcctgtgcatctcaccaatgagatgaaaaactacatgaagaaatcttcctacatgcaagacagtgctatagattcttcttccaaggatcaccactggtcacgtggtaccctgaggcacatctcggagaactcctttgggccatctgggggcctgcgggaaggaagcctgagttctcaagattccaggactgagagtgccagcttgtctcaaagccaggtcaatggtttctttgccagccatttaggtgaccaaacctggcaggaatcacagcatggcagcccttccccatctgtaatatccaaagccaccgagaaagagactttcactgatagtaaccaaagcaaaactaaaaagccaggcatttctgatgtaactgattactcagaccgtggagattcagacatggatgaagccacttactccagcagtcaggatcatcaaacaccaaaacaggaatcttcctcttcagtgaatacatccaacaagatgaattttaaaactttttcttcatcacctcctaagcctggagatatctttgaggttgaactggctaaaaatgataacagcttggggataagtgtcacgggaggtgtgaatacgagtgtcagacatggtggcatttatgtgaaagctgttattccccagggagcagcagagtctgatggtagaattcacaaaggtgatcgcgtcctagctgtcaatggagttagtctagaaggagccacccataagcaagctgtggaaacactgagaaatacaggacaggtggttcatctgttattagaaaagggacaatctccaacatctaaagaacatgtcccggtaaccccacagtgtaccctttcagatcagaatgcccaaggtcaaggcccagaaaaagtgaagaaaacaactcaggtcaaagactacagctttgtcactgaagaaaatacatttgaggtaaaattatttaaaaatagctcaggtctaggattcagtttttctcgagaagataatcttataccggagcaaattaatgccagcatagtaagggttaaaaagctctttcctggacagccagcagcagaaagtggaaaaattgatgtaggagatgttatcttgaaagtgaatggagcctctttgaaaggactatctcagcaggaagtcatatctgctctcaggggaactgctccagaagtattcttgcttctctgcagacctccacctggtgtgctaccggaaattgatactgcgcttttgaccccacttcagtctccagcacaagtacttccaaacagcagtaaagactcttctcagccatcatgtgtggagcaaagcaccagctcagatgaaaatgaaatgtcagacaaaagcaaaaaacagtgcaagtccccatccagaagagacagttacagtgacagcagtgggagtggagaagatgacttagtgacagctccagcaaacatatcaaattcgacctggagttcagctttgcatcagactctaagcaacatggtatcacaggcacagagtcatcatgaagcacccaagagtcaagaagataccatttgtaccatgttttactatcctcagaaaattcccaataaaccagagtttgaggacagtaatccttcccctctaccaccggatatggctcctgggcagagttatcaaccccaatcagaatctgcttcctctagttcgatggataagtatcatatacatcacatttctgaaccaactagacaagaaaactggacacctttgaaaaatgacttggaaaatcaccttgaagactttgaactggaagtagaactcctcattaccctaattaaatcagaaaaaggaagcctgggttttacagtaaccaaaggcaatcagagaattggttgttatgttcatgatgtcatacaggatccagccaaaagtgatggaaggctaaaacctggggaccggctcataaaggttaatgatacagatgttactaatatgactcatacagatgcagttaatctgctccgggctgcatccaaaacagtcagattagttattggacgagttctagaattacccagaataccaatgttgcctcatttgctaccggacataacactaacgtgcaacaaagaggagttgggtttttccttatgtggaggtcatgacagcctttatcaagtggtatatattagtgatattaatccaaggtccgtcgcagccattgagggtaatctccagctattagatgtcatccattatgtgaacggagtcagcacacaaggaatgaccttggaggaagttaacagagcattagacatgtcacttccttcattggtattgaaagcaacaagaaatgatcttccagtggtccccagctcaaagaggtctgctgtttcagctccaaagtcaaccaaaggcaatggttcctacagtgtggggtcttgcagccagcctgccctcactcctaatgattcattctccacggttgctggggaagaaataaatgaaatatcgtaccccaaaggaaaatgttctacttatcagataaagggatcaccaaacttgactctgcccaaagaatcttatatacaagaagatgacatttatgatgattcccaagaagctgaagttatccagtctctgctggatgttgtggatgaggaagcccagaatcttttaaacgaaaataatgcagcaggatactcctgtggtccaggtacattaaagatgaatgggaagttatcagaagagagaacagaagatacagactgcgatggttcacctttacctgagtattttactgaggccaccaaaatgaatggctgtgaagaatattgtgaagaaaaagtaaaaagtgaaagcttaattcagaagccacaagaaaagaagactgatgatgatgaaataacatggggaaatgatgagttgccaatagagagaacaaaccatgaagattctgataaagatcattcctttctgacaaacgatgagctcgctgtactccctgtcgtcaaagtgcttccctctggtaaatacacgggtgccaacttaaaatcagtcattcgagtcctgcggggtttgctagatcaaggaattccttctaaggagctggagaatcttcaagaattaaaacctttggatcagtgtctaattgggcaaactaaggaaaacagaaggaagaacagatataaaaatatacttccctatgatgctacaagagtgcctcttggagatgaaggtggctatatcaatgccagcttcattaagataccagttgggaaagaagagttcgtttacattgcctgccaaggaccactgcctacaactgttggagacttctggcagatgatttgggagcaaaaatccacagtgatagccatgatgactcaagaagtagaaggagaaaaaatcaaatgccagcgctattggcccaacatcctaggcaaaacaacaatggtcagcaacagacttcgactggctcttgtgagaatgcagcagctgaagggctttgtggtgagggcaatgacccttgaagatattcagaccagagaggtgcgccatatttctcatctgaatttcactgcctggccagaccatgatacaccttctcaaccagatgatctgcttacttttatctcctacatgagacacatccacagatcaggcccaatcattacgcactgcagtgctggcattggacgttcagggaccctgatttgcatagatgtggttctgggattaatcagtcaggatcttgattttgacatctctgatttggtgcgctgcatgagactacaaagacacggaatggttcagacagaggatcaatatattttctgctatcaagtcatcctttatgtcctgacacgtcttcaagcagaagaagagcaaaaacagcagcctcagcttctgaagtgacatgaaaagagcctctggatgcatttccatttctctccttaacctccagcagactcctgctctctatccaaaataaagatcacagagcagcaagttcatacaacatgcatgttctcctctatcttagaggggtattcttcttgaaaataaaaaatattgaaatgctgtatttttacagctactttaacctatgataattatttacaaaattttaacactaaccaaacaatgcagatcttagggatgattaaaggcagcatttgatgatagcagacattgttacaaggacatggtgagtctatttttaatgcaccaatcttgtttatagcaaaaatgttttccaatattttaataaagtagttattttataggggatacttgaaaccagtatttaagctttaaatgacagtaatattggcatagaaaaaagtagcaaatgtttactgtatcaatttctaatgtttactatatagaatttcctgtaatatatttatatactttttcatgaaaatggagttatcagttatctgtttgttactgcatcatctgtttgtaatcattatctcactttgtaaataaaaacacaccttaaaacatg

SEQ ID NO: 46 - protein, 2485 aa, Homo sapiens
MHVSLAEALEVRGGPLQEEEIWAVLNQSAESLQELFRKVSLADPAALGFIISPWSLLLLPSGSVSFTDENISNQDLRAFTAPEVLQNQSLTSLSDVEKIHIYSLGMTLYWGADYEVPQSQPIKLGDHLNSILLGMCEDVIYARVSVRTVLDACSAHIRNSNCAPSFSYVKHLVKLVLGNLSGTDQLSCNSEQKPDRSQAIRDRLRGKGLPTGRSSTSDVLDIQKPPLSHQTFLNKGLSKSMGFLSIKDTQDENYFKDILSDNSGREDSENTFSPYQFKTSGPEKKPIPGIDVLSKKKIWASSMDLLCTADRDFSSGETATYRRCHPEAVTVRTSTTPRKKEARYSDGSIALDIFGPQKMDPIYHTRELPTSSAISSALDRIRERQKKLQVLREAMNVEEPVRRYKTYHGDVFSTSSESPSIISSESDFRQVRRSEASKRFESSSGLPGVDETLSQGQSQRPSRQYETPFEGNLINQEIMLKRQEEELMQLQAKMALRQSRLSLYPGDTIKASMLDITRDPLREIALETAMTQRKLRNFFGPEFVKMTIEPFISLDLPRSILTKKGKNEDNRRKVNIMLLNGQRLELTCDTKTICKDVFDMVVAHIGLVEHHLFALATLKDNEYFFVDPDLKLTKVAPEGWKEEPKKKTKATVNFTLFFRIKFFMDDVSLIQHTLTCHQYYLQLRKDILEERMHCDDETSLLLASLALQAEYGDYQPEVHGVSYFRMEHYLPARVMEKLDLSYIKEELPKLHNTYVGASEKETELEFLKVCQRLTEYGVHFHRVHPEKKSQTGILLGVCSKGVLVFEVHNGVRTLVLRFPWRETKKISFSKKKITLQNTSDGIKHGFQTDNSKICQYLLHLCSYQHKFQLQMRARQSNQDAQDIERASFRSLNLQAESVRGFNMGRAISTGSLASSTLNKLAVRPLSVQAEILKRLSCSELSLYQPLQNSSKEKNDKASWEEKPREMSKSYHDLSQASLYPHRKNVIVNMEPPPQTVAELVGKPSHQMSRSDAESLAGVTKLNNSKSVASLNRSPERRKHESDSSSIEDPGQAYVLGMTMHSSGNSSSQVPLKENDVLHKRWSIVSSPEREITLVNLKKDAKYGLGFQIIGGEKMGRLDLGIFISSVAPGGPADLDGCLKPGDRLISVNSVSLEGVSHHAAIEILQNAPEDVTLVISQPKEKISKVPSTPVHLTNEMKNYMKKSSYMQDSAIDSSSKDHHWSRGTLRHISENSFGPSGGLREGSLSSQDSRTESASLSQSQVNGFFASHLGDQTWQESQHGSPSPSVISKATEKETFTDSNQSKTKKPGISDVTDYSDRGDSDMDEATYSSSQDHQTPKQESSSSVNTSNKMNFKTFSSSPPKPGDIFEVELAKNDNSLGISVTGGVNTSVRHGGIYVKAVIPQGAAESDGRIHKGDRVLAVNGVSLEGATHKQAVETLRNTGQVVHLLLEKGQSPTSKEHVPVTPQCTLSDQNAQGQGPEKVKKTTQVKDYSFVTEENTFEVKLFKNSSGLGFSFSREDNLIPEQINASIVRVKKLFPGQPAAESGKIDVGDVILKVNGASLKGLSQQEVISALRGTAPEVFLLLCRPPPGVLPEIDTALLTPLQSPAQVLPNSSKDSSQPSCVEQSTSSDENEMSDKSKKQCKSPSRRDSYSDSSGSGEDDLVTAPANISNSTWSSALHQTLSNMVSQAQSHHEAPKSQEDTICTMFYYPQKIPNKPEFEDSNPSPLPPDMAPGQSYQPQSESASSSSMDKYHIHHISEPTRQENWTPLKNDLENHLEDFELEVELLITLIKSEKGSLGFTVTKGNQRIGCYVHDVIQDPAKSDGRLKPGDRLIKVNDTDVTNMTHTDAVNLLRAASKTVRLVIGRVLELPRIPMLPHLLPDITLTCNKEELGFSLCGGHDSLYQVVYISDINPRSVAAIEGNLQLLDVIHYVNGVSTQGMTLEEVNRALDMSLPSLVLKATRNDLPVVPSSKRSAVSAPKSTKGNGSYSVGSCSQPALTPNDSFSTVAGEEINEISYPKGKCSTYQIKGSPNLTLPKESYIQEDDIYDDSQEAEVIQSLLDVVDEEAQNLLNENNAAGYSCGPGTLKMNGKLSEERTEDTDCDGSPLPEYFTEATKMNGCEEYCEEKVKSESLIQKPQEKKTDDDEITWGNDELPIERTNHEDSDKDHSFLTNDELAVLPVVKVLPSGKYTGANLKSVIRVLRGLLDQGIPSKELENLQELKPLDQCLIGQTKENRRKNRYKNILPYDATRVPLGDEGGYINASFIKIPVGKEEFVYIACQGPLPTTVGDFWQMIWEQKSTVIAMMTQEVEGEKIKCQRYWPNILGKTTMVSNRLRLALVRMQQLKGFVVRAMTLEDIQTREVRHISHLNFTAWPDHDTPSQPDDLLTFISYMRHIHRSGPIITHCSAGIGRSGTLICIDVVLGLISQDLDFDISDLVRCMRLQRHGMVQTEDQYIFCYQVILYVLTRLQAEEEQKQQPQLLK

SEQ ID NO: 47 - DNA, 20 bp, Artificial Sequence
  Synthetic Sequence
acgtgcatattaccggctgg

SEQ ID NO: 48 - DNA, 20 bp, Artificial Sequence
  Synthetic Sequence
gagaaatgatgaagccaagg

SEQ ID NO: 49 - DNA, 20 bp, Artificial Sequence
  Synthetic Sequence
gttggctctgaggcacttca

SEQ ID NO: 50 - DNA, 20 bp, Artificial Sequence
  Synthetic Sequence
tttgtctctctcggattcgg

SEQ ID NO: 51 - DNA, 20 bp, Artificial Sequence
  Synthetic Sequence
gccaaagaaattcctcagtt

SEQ ID NO: 52 - DNA, 20 bp, Artificial Sequence
  Synthetic Sequence
aaggatgccagcaataagga

SEQ ID NO: 53 - DNA, 20 bp, Artificial Sequence
  Synthetic Sequence
ggtcttcaatggatgaggag

SEQ ID NO: 54 - DNA, 20 bp, Artificial Sequence
  Synthetic Sequence
gtggtgatccttggaagaag

SEQ ID NO: 55 - DNA, 20 bp, Artificial Sequence
  Synthetic Sequence
tccactcccactgctgtcac

SEQ ID NO: 56 - DNA, 20 bp, Artificial Sequence
  Synthetic Sequence
ttctctgattgcctttggtt

SEQ ID NO: 57 - DNA, 20 bp, Artificial Sequence
  Synthetic Sequence
gcaactcatcatttccccat

SEQ ID NO: 58 - DNA, 20 bp, Artificial Sequence
  Synthetic Sequence
ccagaggctcttttcatgtc

SEQ ID NO: 59 - DNA, 20 bp, Artificial Sequence
  Synthetic Sequence
gcatccagaggctcttttca

SEQ ID NO: 60 - DNA, 20 bp, Artificial Sequence
  Synthetic Sequence
gctggaggttaaggagagaa

SEQ ID NO: 61 - DNA, 20 bp, Artificial Sequence
  Synthetic Sequence
tttggatagagagcaggagt

SEQ ID NO: 62 - DNA, 20 bp, Artificial Sequence
  Synthetic Sequence
tttcaagaagaataccccta

SEQ ID NO: 63 - DNA, 20 bp, Artificial Sequence
  Synthetic Sequence
gctgcctttaatcatcccta

SEQ ID NO: 64 - DNA, 20 bp, Artificial Sequence
  Synthetic Sequence
actggtttcaagtatcccct

SEQ ID NO: 65 - DNA, 1480 bp, Mus musculus
  CDS (50)..(1033)
  ref <303>: JOURNAL: J. Immunol.
  ref <304>: VOLUME: 148
  ref <306>: PAGES: 1274-1297
  ref <307>: DATE: 1992-02-15
  ref <308>: DATABASE ACCESSION NUMBER: M83649/Genbank
  ref <309>: DATABASE ENTRY DATE: 1994-04-18
gccgcaggctgcccacacaggccgcccgctgttttcccttgctgcagacatgctgtggatctgggctgtcctgcctctggtgcttgctggctcacagttaagagttcatactcaaggtactaatagcatctccgagagtttaaagctgaggaggcgggttcatgaaactgataaaaactgctcagaaggattatatcaaggaggcccattttgctgtcaaccatgccaacctggtaaaaaaaaagttgaggactgcaaaatgaatgggggtacaccaacctgtgccccatgcacagaagggaaggagtacatggacaagaaccattatgctgataaatgcagaagatgcacactctgcgatgaagagcatggtttagaagtggaaacaaactgcaccctgacccagaataccaagtgcaagtgcaaaccagacttctactgcgattctcctggctgtgaacactgtgttcgctgcgcctcgtgtgaacatggaacccttgagccatgcacagcaaccagcaatacaaactgcaggaaacaaagtcccagaaatcgcctatggttgttgaccatccttgttttgttaattccacttgtatttatatatcgaaagtaccggaaaagaaagtgctggaaaaggagacaggatgaccctgaatctagaacctccagtcgtgaaaccataccaatgaatgcctcaaatcttagcttgagtaaatacatcccgagaattgctgaagacatgacaatccaggaagctaaaaaatttgctcgagaaaataacatcaaggagggcaagatagatgagatcatgcatgacagcatccaagacacagctgagcagaaagtccagctgctcctgtgctggtaccaatctcatgggaagagtgatgcatatcaagatttaatcaagggtctcaaaaaagccgaatgtcgcagaaccttagataaatttcaggacatggtccagaaggaccttggaaaatcaaccccagacactggaaatgaaaatgaaggacaatgtctggagtgaaaactacctcagttccagccatgaagagaggagagagcctgccacccatgatggaaacaaaatgaatgccaactgtattgacattggcaactcctggtgtgttctctttgccagcaaatggtagttgatactcagtgagggtcaaatgactagcaggttccagggactgcttctgttattctctgcagttgctgagatgaaccattttctctgtctactgcaatttttacattcaaatgtccatgaaatttgtattaaatgtgaagtggaatctgcagtgtttgtgtttatattcatatactatgaactgaggagaattataaactgaaacaaatactcgcagttaattgaagaccttccattgatggacagttcttttcctctctatatggaaatgtataatagaagaaataatttttaaattaaagtatctctttttgcatttca

SEQ ID NO: 66 - protein, 327 aa, Mus musculus
MLWIWAVLPLVLAGSQLRVHTQGTNSISESLKLRRRVHETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYRKYRKRKCWKRRQDDPESRTSSRETIPMNASNLSLSKYIPRIAEDMTIQEAKKFARENNIKEGKIDEIMHDSIQDTAEQKVQLLLCWYQSHGKSDAYQDLIKGLKKAECRRTLDKFQDMVQKDLGKSTPDTGNENEGQCLE

SEQ ID NO: 67 - DNA, 20 bp, Artificial Sequence
  Synthetic Sequence
gcagcaagggaaaacagcgg

SEQ ID NO: 68 - DNA, 20 bp, Artificial Sequence
  Synthetic Sequence
ccacagcatgtctgcagcaa

SEQ ID NO: 69 - DNA, 20 bp, Artificial Sequence
  Synthetic Sequence
tttcatgaacccgcctcctc

SEQ ID NO: 70 - DNA, 20 bp, Artificial Sequence
  Synthetic Sequence
gggtcagggtgcagtttgtt

SEQ ID NO: 71 - DNA, 20 bp, Artificial Sequence
  Synthetic Sequence
gaggcgcagcgaacacagtg

SEQ ID NO: 72 - DNA, 20 bp, Artificial Sequence
  Synthetic Sequence
cataggcgatttctgggact

SEQ ID NO: 73 - DNA, 20 bp, Artificial Sequence
  Synthetic Sequence
tccagcactttcttttccgg

SEQ ID NO: 74 - DNA, 20 bp, Artificial Sequence
  Synthetic Sequence
ggtttcacgactggaggttc

SEQ ID NO: 75 - DNA, 20 bp, Artificial Sequence
  Synthetic Sequence
cttcagcaattctcgggatg

SEQ ID NO: 76 - DNA, 20 bp, Artificial Sequence
  Synthetic Sequence
gccctccttgatgttatttt

SEQ ID NO: 77 - DNA, 20 bp, Artificial Sequence
  Synthetic Sequence
ggtaccagcacaggagcagc

SEQ ID NO: 78 - DNA, 20 bp, Artificial Sequence
  Synthetic Sequence
cggcttttttgagacccttg

SEQ ID NO: 79 - DNA, 20 bp, Artificial Sequence
  Synthetic Sequence
gtgtctggggttgattttcc

SEQ ID NO: 80 - DNA, 20 bp, Artificial Sequence
  Synthetic Sequence
tctcctctcttcatggctgg

SEQ ID NO: 81 - DNA, 20 bp, Artificial Sequence
  Synthetic Sequence
ggcattcattttgtttccat

SEQ ID NO: 82 - DNA, 20 bp, Artificial Sequence
  Synthetic Sequence
tccctggaacctgctagtca

SEQ ID NO: 83 - DNA, 20 bp, Artificial Sequence
  Synthetic Sequence
tcagcaactgcagagaataa

SEQ ID NO: 84 - DNA, 20 bp, Artificial Sequence
  Synthetic Sequence
gcagattccacttcacattt

SEQ ID NO: 85 - DNA, 20 bp, Artificial Sequence
  Synthetic Sequence
aaggtcttcaattaactgcg

SEQ ID NO: 86 - DNA, 20 bp, Artificial Sequence
  Antisense Oligonucleotide
tccgtcatcgctcctcaggg

SEQ ID NO: 87 - DNA, 20 bp, Artificial Sequence
  Antisense Oligonucleotide
atgcattctgcccccaagga

SEQ ID NO: 88 - DNA, 29 bp, Artificial Sequence
  PCR Primer
tcatgacactaagtcaagttaaaggcttt

SEQ ID NO: 89 - DNA, 26 bp, Artificial Sequence
  PCR Primer
tcttggacattgtcattcttgatctc

SEQ ID NO: 90 - DNA, 31 bp, Artificial Sequence
  PCR Probe
attttggcttcattgacaccattctttcgaa

SEQ ID NO: 91 - DNA, 21 bp, Artificial Sequence
  PCR Primer
caacggatttggtcgtattgg

SEQ ID NO: 92 - DNA, 26 bp, Artificial Sequence
  PCR Primer
ggcaacaatatccactttaccagagt

SEQ ID NO: 93 - DNA, 21 bp, Artificial Sequence
  PCR Probe
cgcctggtcaccagggctgct

SEQ ID NO: 94 - DNA, 2165 bp, Homo sapiens
  CDS (1782)...(1813)
aagcttttttggctacatttttttatttgtaaagtaagtttaataatcactcatctcactgggctataatgataagtattaagtaaggaagatccacatatgtgagttgctggcttataattcacactcaagagatactgattttgtcaattgtcctttcccctttttttctctcttccctccttccattccttcttcccttacctctcctttccttccctcacaccccttttccttccttctttttacatttttttatttaaatgaacttttcattttggaatagttttaggatttcaaaaaatttgcagagataatacagagaatgcccatataccatcctccttatcccacttctttttgtgtctattagatgctcagagtgtgtgcacaaggctggcacacccagggtcttcctcatggcactaacagtctactgaaaggtggaacagagacaagcctatcaacacctacaagactggtggtaagtgcagtgacagatgcaaaacacagggtgatggaaagccctcaggagggtaacctaacctagatttgagggcccaaacaggctccagaagaaaatgtcaactgagaggaagcctgaaggatgaacagtgggctaagcaaagggttattaatgtgttattaatgggttgaatctaattgggaagggagagaggttgcagagtgaggtgcagagcttggtggacgatgccaaaggaatactgaaacctttagtgtgtccagtctggaactgcatccaaattcaggttcagtaatgatgtcattatccaaacataccttctgtaaaattcatgctaaactacctaagagctatctaccgttccaaagcaatagtgactttgaacagtgttcaccagagcacgaaagaattacaagatttttttttaaagaaaattggccaggaaataatgagtaacgaaggacaggaagtaattgtgaatgtttaatatagctggggctatgcgatttggcttaagttgttagctttgttttcctcttgagaaataaaaactaaggggccctcccttttcagagccctatggcgcaacatctgtactttttcatatggttaactgtccattccaggaacgtctgtgagcctctcatgttgcagccacaacatggacagcccagtcaaatgccccgcaagtctttctctgagtgactccagcaattagccaaggctcctgtacccaggcaggacctctgcgctctgagctccattctccttcaagacctccccaacttcccaggttgaactacagcagaagcctttagaaagggcaggaggccggctctcgaggtcctcacctgaagtgagcatgccagccactgcaggaacgccccgggacaggaatgcccatttgtgcaacgaaccctgactccttcctcaccctgacttctccccctccctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggtgagccctctcctgcccgggtggaggcttaccccgtcttagtcccggggataggcaaagtggggcgggcgcgggacgcgtgcgggattgcggcggcagcggcgcacgcgggcacctgggagcggcgggctgctgcgggaggcgttggagactggctcccgggggctgttaggaccttccctcaggcccgggtgctcagaacgctggaggacttgcttttcttgggccttgatgcgaagtgctgaccccgctgggcaggcggggcagctccggcgctcctcggagaccactgcgctccacgttgaggtgggcgtggggggcggacaggaattgaagcggaagtctgggaagctt

SEQ ID NO: 95 - DNA, 623 bp, Homo sapiens
  CDS (155)...(320)
ctttcttggagagagaaatctgaaagacagtggagccctcacattgtctttgcctgtgcacagcagatactgccaattttgggtgggttacactggtttacacgttgcttacttcagaaatcaataaaattctcttcatgcttttattttacaggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtatgttacacaaaacatccagagattacagtgaaagtcacagttaggagtagcacatagtaatcatgactataataattttacagtttttggttcccctatattatataacataactgagagaaaaacaactatgaaattattttccaaagatgagttttatttatatttatcatgcttatttgatgtggttatggataaatttaatttacaagtgacatgcacctctgaaatgagaagactggtctatttggctccatttttttctaagcaaaaatgactcatttgtgaatatgaaagctt

SEQ ID NO: 96 - DNA, 924 bp, Homo sapiens
  CDS (146)...(283)
cccccattgtatttatatctcattagcctaccccccctccccttgtgttttagaagagttttattgtctgtcatccctctatacttcccaccctgttacctgcccgtgtcctgttcaaacacttgctccttttttccttgggcaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggtaagagtcttaaaatgcaattgaaagaggccaatcttggaatttcatgtagaaccatttataagacaatttgaaattggggcctactgtggtgctatgttgacacacaggaaagggaaggacaggtggctagggtaccgcagaaccaggtgccgagctaactactggtctagacctttatgagtaagtctaggcaattcttccagatataggagaatgagtaaatatgaaccctaggaacagggttcatcagctcaaatcaaaagctcagaaattattttttttctggccttgacttacgcttatataatggtgctcgttcatggccagaaaaattcagaagcctgcagctgcagatacaaggacacagaaatccaaaaggtaggtaggaatgctccccctttctttggggatttagtttgtcctgataattccatctggggacctgaattttcatggatatctcaatgtattctaaggacccagattgaagtataacagaagtgtttctagttttgtttgacatgaagaaacctctggacataaagcttttcccatccctattcagcctataaagcaatgtttctcatccctggcagtgcattagaattgcttagggagctactaaaatgtttcatgtt

SEQ ID NO: 97 - DNA, 368 bp, Homo sapiens
  CDS (145)...(253)
aacactgactgtattactggtgtcatgctgtgactgttgatataagcagtggatctcaaaaatccatgcagctcctgcccaccattttcatagtctgcttataattagccgctataactaatagtttccaaactgattttctaggcttataagtggaaatatactgcacccggacccagaataccaagtgcagatgtaaaccaaactttttttgtaactctactgtatgtgaacactgtgacccttgcaccaagtaagttttagtctttctctgattaaaacactagatataacatgagagttatcattttcctagggaagtaacactgactgagagttaagaattagggttctagtcctgctttgcc

SEQ ID NO: 98 - DNA, 855 bp, Homo sapiens
  CDS (551)...(613)
  CDS (766)...(828)
aactcagacaacctgattgtgaatgtttgtctgtctgaaggaaatcacacatgaacctcttgagtctcctgatcaccaccggttgctaaaagtggcagcctctaagggcagctgagtaccctccctgagctacatcatgggcgtggctatcacctggccattttcttggtctataggaattttttgaaattacttttgacagtttattttaagagctagtttaagctataggatttacgtgttcagtttattactaggtttaagtttatttttgtatccacttcatctctcttgtgtgtcactattttcctatcttcctttaactcttgaaatcttaagacagtcattccttatgatatttttcatccagccatccaaattatattaacttgtgccagctttagatactaatttagaaatatttgaaggaatacgtttgccagagatgcaaagatgaataaaatggcccctaatttacaaagtgccattgaaaattataaaggaattattctgccaggcttttgaatttctcctgtatttttttttctagatgtgtacatggaatcatcaaggaatgcacactcaccagcaacaccaagtgcaaagaggaaggtaattatttttttacggttatattctcctttcccccaaccccatggaaagatgtgaagaaaaaccaatcactcttgattagtagaaagtcctttatttaatcttaaagattgcttattttcatataaaatgtccaatgttccaacctacaggatccagatctatcttggggtggctttgtcttcttcttttgccaattccactaattgtttggggtaagttcttgctttgttcaaactgcag

SEQ ID NO: 99 - DNA, 338 bp, Homo sapiens
  CDS (79)...(161)
tcttagtgtgaaagtatgttctcacatgcattctacaaggctgagacctgagttgataaaatttctttgttctttcagtgaagagaaaggaagtacagaaaacatgcagaaagcacagaaaggaaaaccaaggttctcatgaatctccaaccttatatcctgtaggtattgaaataggtatcagctttccttgaaaagaaaaatagagaaattagtgatttggctttttgttacttccttttacttttttgtttcttgtttgtttcattttgtttgagatggagtcttgctccatagcccaggctggagtgcaggggtgcaatcatggctcactgcag

SEQ ID NO: 100 - DNA, 734 bp, Homo sapiens
  CDS (255)...(279)
gaattctttaggtttcttgcctttaaaaactaagacaatattgcttagtttctggcaaggccggaacctttcagaataaaaattgaatggtaaaagtaaccttcttaatcacttaatctagcttcctaattttatacatcaagcaactgattgtacttctttctgaattaaggaaaaattagaagttcacatttagaatattctaaagatatatttttatttgtctttctctgcttccattttttgctttctaggaaacagtggcaataaatttatctggtaaggcttttatcattttatttcatagagatggcatcctttagagtaataggccaatttcagagtaaaataatgttactaatttcagtgacatattatgggatcttgttatttctcatacattctacctgctcagcataaagcatttatcaggcagtttgtttaaatttataatgagtactcatagttaaaaataatcaagtaacaataagacacaatagtctgaggcttaagaaacttttccttcataatcagctagatgtattacagaactcctgcctaaaaagatctagaggttaaagtgtactgtagactcaggtattatcagtgtacccaactctataacaacatacatgattccattcagtggttctttgatctgtgatttagagataagatgatcataaactctttgcttatacttttagatttgtgggtcattgatcattggatcc

SEQ ID NO: 101 - DNA, 1840 bp, Homo sapiens
  CDS (95)...(426)
ttctgaagtactataaaagagaaataaacatggttttcactaatgggaatttcatttagaaaaacaaattttcagactattttctatttttcagatgttgacttgtgtaaatatatcaccactattgctggagtcatgtcactatgtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataact

SEQ ID NO: 102 - DNA, 836 bp, Homo sapiens
  CDS (1)...(399)
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatgatgtgaacatggaatcatcaaggaatgcacactcaccagcaacaccaagtgcaaagaggaagtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 103 - DNA, 20 bp, Artificial Sequence
  Antisense Oligonucleotide
tgaggaaggagtcagggttc

SEQ ID NO: 104 - DNA, 20 bp, Artificial Sequence
  Antisense Oligonucleotide
ggtggtcaggaggatgggaa

SEQ ID NO: 105 - DNA, 20 bp, Artificial Sequence
  Antisense Oligonucleotide
agccagtctccaacgcctcc

SEQ ID NO: 106 - DNA, 20 bp, Artificial Sequence
  Antisense Oligonucleotide
tgccccgcctgcccagcggg

SEQ ID NO: 107 - DNA, 20 bp, Artificial Sequence
  Antisense Oligonucleotide
acacctgtgtgtcactcttg

SEQ ID NO: 108 - DNA, 20 bp, Artificial Sequence
  Antisense Oligonucleotide
gccaagtcactcgtaaaccg

SEQ ID NO: 109 - DNA, 20 bp, Artificial Sequence
  Antisense Oligonucleotide
aatcctccgaagtgaaagag

SEQ ID NO: 110 - DNA, 20 bp, Artificial Sequence
  Antisense Oligonucleotide
atgcccagcatggttgttga

SEQ ID NO: 111 - DNA, 20 bp, Artificial Sequence
  Antisense Oligonucleotide
acgtaagaaccagaggtagg

SEQ ID NO: 112 - DNA, 20 bp, Artificial Sequence
  Antisense Oligonucleotide
ttttggacgataatctagca

SEQ ID NO: 113 - DNA, 20 bp, Artificial Sequence
  Antisense Oligonucleotide
ttcctttcacctggaggaca

SEQ ID NO: 114 - DNA, 20 bp, Artificial Sequence
  Antisense Oligonucleotide
cagtccctagctttcctttc

SEQ ID NO: 115 - DNA, 20 bp, Artificial Sequence
  Antisense Oligonucleotide
agccatgtccttcatcacac

SEQ ID NO: 116 - DNA, 20 bp, Artificial Sequence
  Antisense Oligonucleotide
gggtcacagtgttcacatac

SEQ ID NO: 117 - DNA, 20 bp, Artificial Sequence
  Antisense Oligonucleotide
gttgctggtgagtgtgcatt

SEQ ID NO: 118 - DNA, 20 bp, Artificial Sequence
  Antisense Oligonucleotide
acttcctttctcttcaccca

SEQ ID NO: 119 - DNA, 20 bp, Artificial Sequence
  Antisense Oligonucleotide
tggttttcctttctgtgctt

SEQ ID NO: 120 - DNA, 20 bp, Artificial Sequence
  Antisense Oligonucleotide
tttaaggttggagattcatg

SEQ ID NO: 121 - DNA, 20 bp, Artificial Sequence
  Antisense Oligonucleotide
gatttaaggttggagattca

SEQ ID NO: 122 - DNA, 20 bp, Artificial Sequence
  Antisense Oligonucleotide
ccactgtttcaggatttaag

SEQ ID NO: 123 - DNA, 20 bp, Artificial Sequence
  Antisense Oligonucleotide
gtcaacatcagataaattta

SEQ ID NO: 124 - DNA, 20 bp, Artificial Sequence
  Antisense Oligonucleotide
tttactcaagtcaacatcag

SEQ ID NO: 125 - DNA, 20 bp, Artificial Sequence
  Antisense Oligonucleotide
ttagtgtcatgactccagca

SEQ ID NO: 126 - DNA, 20 bp, Artificial Sequence
  Antisense Oligonucleotide
aacttgacttagtgtcatga

SEQ ID NO: 127 - DNA, 20 bp, Artificial Sequence
  Antisense Oligonucleotide
tacgaagcagttgaactttc

SEQ ID NO: 128 - DNA, 20 bp, Artificial Sequence
  Antisense Oligonucleotide
ttgagatctttaatcaatgt

SEQ ID NO: 129 - DNA, 20 bp, Artificial Sequence
  Antisense Oligonucleotide
gtccttgaggatgatagtct

SEQ ID NO: 130 - DNA, 20 bp, Artificial Sequence
  Antisense Oligonucleotide
ttggatttcatttctgaagt

SEQ ID NO: 131 - DNA, 20 bp, Artificial Sequence
  Antisense Oligonucleotide
tttttcactctagaccaagc

SEQ ID NO: 132 - DNA, 20 bp, Artificial Sequence
  Antisense Oligonucleotide
aagcagtatttacagccagc

SEQ ID NO: 133 - DNA, 20 bp, Artificial Sequence
  Antisense Oligonucleotide
tcagcgctaataaatgataa

SEQ ID NO: 134 - DNA, 20 bp, Artificial Sequence
  Antisense Oligonucleotide
ctcttcagcgctaataaatg

SEQ ID NO: 135 - DNA, 20 bp, Artificial Sequence
  Antisense Oligonucleotide
atgccactgcatttactctt

SEQ ID NO: 136 - DNA, 20 bp, Artificial Sequence
  Antisense Oligonucleotide
catgccactgcatttactct

SEQ ID NO: 137 - DNA, 20 bp, Artificial Sequence
  Antisense Oligonucleotide
acattcatactacagaatca

SEQ ID NO: 138 - DNA, 20 bp, Artificial Sequence
  Antisense Oligonucleotide
catacactgattacattcat

SEQ ID NO: 139 - DNA, 20 bp, Artificial Sequence
  Antisense Oligonucleotide
ttacataaatatgatcttct

SEQ ID NO: 140 - DNA, 20 bp, Artificial Sequence
  Antisense Oligonucleotide
gaggtagagccttatttaaa

SEQ ID NO: 141 - DNA, 20 bp, Artificial Sequence
  Antisense Oligonucleotide
gtataatatgacaccaataa

SEQ ID NO: 142 - DNA, 20 bp, Artificial Sequence
  Antisense Oligonucleotide
aatattgtataatatgacac

SEQ ID NO: 143 - DNA, 20 bp, Artificial Sequence
  Antisense Oligonucleotide
gtgaattcacaattgaaata

SEQ ID NO: 144 - DNA, 20 bp, Artificial Sequence
  Antisense Oligonucleotide
attataatttaatgttttct

SEQ ID NO: 145 - DNA, 20 bp, Artificial Sequence
  Antisense Oligonucleotide
tactctcctgctcaaaatgc

SEQ ID NO: 146 - DNA, 20 bp, Artificial Sequence
  Antisense Oligonucleotide
tggtggactattaagtattt

SEQ ID NO: 147 - DNA, 20 bp, Artificial Sequence
  Antisense Oligonucleotide
agagcagttagtatctccaa

SEQ ID NO: 148 - DNA, 20 bp, Artificial Sequence
  Antisense Oligonucleotide
caaagctactttctctgaga

SEQ ID NO: 149 - DNA, 20 bp, Artificial Sequence
  Antisense Oligonucleotide
gacatgtcacaaagctactt

SEQ ID NO: 150 - DNA, 20 bp, Artificial Sequence
  Antisense Oligonucleotide
ttatcatctttgattgcaaa

SEQ ID NO: 151 - DNA, 20 bp, Artificial Sequence
  Antisense Oligonucleotide
atgggacattattgaacatt

SEQ ID NO: 152 - DNA, 20 bp, Artificial Sequence
  Antisense Oligonucleotide
attcacatttaatacaaact

SEQ ID NO: 153 - DNA, 20 bp, Artificial Sequence
  Antisense Oligonucleotide
atataaatattatttcttaa

SEQ ID NO: 154 - DNA, 20 bp, Artificial Sequence
  Antisense Oligonucleotide
ctatgtgctactcctaactg

SEQ ID NO: 155 - DNA, 20 bp, Artificial Sequence
  Antisense Oligonucleotide
tgattactatgtgctactcc

SEQ ID NO: 156 - DNA, 20 bp, Artificial Sequence
  Antisense Oligonucleotide
tataaataaaactcatcttt

SEQ ID NO: 157 - DNA, 20 bp, Artificial Sequence
  Antisense Oligonucleotide
cttccctttcctgtgtgtca

SEQ ID NO: 158 - DNA, 20 bp, Artificial Sequence
  Antisense Oligonucleotide
taccctagccacctgtcctt

SEQ ID NO: 159 - DNA, 20 bp, Artificial Sequence
  Antisense Oligonucleotide
ctggaagaattgcctagact

SEQ ID NO: 160 - DNA, 20 bp, Artificial Sequence
  Antisense Oligonucleotide
atatttactcattctcctat

SEQ ID NO: 161 - DNA, 20 bp, Artificial Sequence
  Antisense Oligonucleotide
atgtccagaggtttcttcat

SEQ ID NO: 162 - DNA, 20 bp, Artificial Sequence
  Antisense Oligonucleotide
agaaacattgctttataggc

SEQ ID NO: 163 - DNA, 20 bp, Artificial Sequence
  Antisense Oligonucleotide
atgacaccagtaatacagtc

SEQ ID NO: 164 - DNA, 20 bp, Artificial Sequence
  Antisense Oligonucleotide
tttgagatccactgcttata

SEQ ID NO: 165 - DNA, 20 bp, Artificial Sequence
  Antisense Oligonucleotide
gtttggaaactattagttat

SEQ ID NO: 166 - DNA, 20 bp, Artificial Sequence
  Antisense Oligonucleotide
atgtgtgatttccttcagac

SEQ ID NO: 167 - DNA, 20 bp, Artificial Sequence
  Antisense Oligonucleotide
atcataaggaatgactgtct

SEQ ID NO: 168 - DNA, 20 bp, Artificial Sequence
  Antisense Oligonucleotide
aatggcactttgtaaattag

SEQ ID NO: 169 - DNA, 20 bp, Artificial Sequence
  Antisense Oligonucleotide
tataattttcaatggcactt

SEQ ID NO: 170 - DNA, 20 bp, Artificial Sequence
  Antisense Oligonucleotide
cagaataattcctttataat

SEQ ID NO: 171 - DNA, 20 bp, Artificial Sequence
  Antisense Oligonucleotide
ccatgttcacatctagaaaa

SEQ ID NO: 172 - DNA, 20 bp, Artificial Sequence
  Antisense Oligonucleotide
tctcttcactgaaagaacaa

SEQ ID NO: 173 - DNA, 20 bp, Artificial Sequence
  Antisense Oligonucleotide
aggaaagctgatacctattt

SEQ ID NO: 174 - DNA, 20 bp, Artificial Sequence
  Antisense Oligonucleotide
catctctatgaaataaaatg

SEQ ID NO: 175 - DNA, 20 bp, Artificial Sequence
  Antisense Oligonucleotide
ggaaaagtttcttaagcctc

SEQ ID NO: 176 - DNA, 20 bp, Artificial Sequence
  Antisense Oligonucleotide
ttatctctaaatcacagatc

SEQ ID NO: 177 - DNA, 20 bp, Artificial Sequence
  Antisense Oligonucleotide
aaagagaaaaccagaaatac

SEQ ID NO: 178 - DNA, 20 bp, Artificial Sequence
  Antisense Oligonucleotide
gttagagaaaaggaagacaa

SEQ ID NO: 179 - DNA, 20 bp, Artificial Sequence
  Antisense Oligonucleotide
atgttcacatcatgtccttc

SEQ ID NO: 180 - DNA, 20 bp, Artificial Sequence
  Antisense Oligonucleotide
tcgatctccttttatgcccg